(19) United States
(12) Patent Application Publication
(10) Pub. No.: US 2009/0145441 A1
(43) Pub. Date: Jun. 11, 2009

(54) DELAYED RESISTANCE NASAL DEVICES AND METHODS OF USE

(76) Inventors: Rajiv Doshi, Los Altos, CA (US); Bryan Loomas, Los Gatos, CA (US); Arthur Ferdinand, San Jose, CA (US); Eric Meyer, Seattle, WA (US); Arthur G. Sandoval, San Francisco, CA (US); Jeffrey W. Servaites, San Francisco, CA (US); Jonathan Patrick Summers, Redwood City, CA (US)

Correspondence Address: SHAY GLENN LLP, 2755 CAMPUS DRIVE, SUITE 210, SAN MATEO, CA, 94403, US

(21) Appl. No.: 12/329,895

(22) Filed: Dec. 8, 2008

Related U.S. Application Data
(60) Provisional application No. 61/012,016, filed on Dec. 6, 2007.

(51) Int. Cl. A61M 15/08

(52) U.S. Cl. 128/207.18

(57) ABSTRACT

Delayed resistance nasal devices include an airflow resistor that is configured to normally have a higher resistance to exhalation than inhalation, but the higher resistance to exhalation may be suspended, or delayed by activation of an airflow resistor bypass. Activation of an airflow resistor bypass bypasses or decreases the effect of the airflow resistor on nasal airflow through the nasal device, decreasing the resistance to exhalation. Methods of decreasing, suspending, or delaying the onset of the inhibition of the exhalation through such nasal respiratory devices are described.

### CROSS-REFERENCE TO RELATED APPLICATION

This application claims priority to U.S. Provisional Patent Application Ser. No. 61/012,016, (titled “DELAYED RESISTANCE NASAL DEVICES AND METHODS OF USE”) filed on Dec. 6, 2007, which is herein incorporated by reference in its entirety.

### BACKGROUND OF THE INVENTION

Nasal respiratory devices may be worn to treat many medical conditions, such as sleep disordered breathing (including snoring, sleep apnea, etc.), Cheyne Stokes breathing, UARS, COPD, hypertension, asthma, GERD, heart failure, and other respiratory and sleep conditions. Devices that provide a greater resistance to exhalation than to inhalation may be particularly useful, and may be worn by a subject when the subject is either awake or asleep. Indeed, many subjects may apply a nasal device before falling to sleep, so that the device may provide therapeutic benefits during sleep. However, some subject's may have difficulty acclimating to the increased resistance to exhalation, particularly when falling asleep.

Examples of nasal respiratory devices have been well-described in the following US patent applications, each of which is incorporated herein in its entirety: U.S. patent application Ser. No. 11/298,640 (titled “NASAL RESPIRATORY DEVICES”) filed Dec. 8, 2005; U.S. patent application Ser. No. 11/298,339 (titled “RESPIRATORY DEVICES”) filed Dec. 8, 2005; U.S. patent application Ser. No. 11/298,362 (titled “METHODS OF TREATING RESPIRATORY DISORDERS”) filed Dec. 8, 2005; U.S. patent application Ser. No. 11/805,496 (titled “NASAL RESPIRATORY DEVICES”) filed May 22, 2007; U.S. patent application Ser. No. 11/811,339 (titled “NASAL DEVICES”) filed Jun. 7, 2007; U.S. patent application Ser. No. 11/759,916 (titled “LAYERED NASAL DEVICES”) filed Jun. 7, 2007; and U.S. patent application Ser. No. 11/811,401 (titled “NASAL RESPIRATORY DEVICES FOR POSITIVE END-EXPIRATORY PRESSURE”) filed Jun. 7, 2007.

These nasal respiratory devices are adapted to be removably secured in communication with a nasal cavity, and may include a passageway with an opening at a proximal end and an opening at a distal end, a valve (or airflow resistor) in communication with the passageway, and a holdfast in communication with the outer walls forming the passageway. The holdfast is configured to removably secure the respiratory device within (or over or around) the nasal cavity. The airflow resistor (which may be a valve) is typically configured to provide greater resistance during exhalation than during inhalation.

When wearing these nasal devices, some individuals may benefit from a period of adjustment during which they can acclimate to the feel of the nasal device and its effect on their nasal breathing. For example, a subject preparing to wear the device while sleeping may more easily fall asleep once he or she has gotten used to the nasal devices. With any of the previously described nasal respiration devices, the resistance to exhalation is typically felt immediately upon applying the device. Thus, it would be beneficial to provide nasal devices and methods of using and making nasal devices that allow control of the onset of resistance to exhalation, suspension of the resistance to exhalation, or delay of the resistance to exhalation (and/or inhalation), allowing time for the subject to acclimate to the feel of the device before the airflow resistance is completely engaged. It would also be beneficial to provide devices that allow the subject (or another person) to temporarily decrease the resistance through the device for some period of time (e.g., when falling asleep wearing the device).

Nasal devices configured to delay (or suspended) a high baseline resistance that may address the issues raised above are described and illustrated, including methods of using and methods of forming such devices.

### SUMMARY OF THE INVENTION

Described herein are nasal devices that include an airflow resistor to inhibit nasal exhalation more than inhalation and an airflow resistor bypass to suspend operation of the airflow resistor. Thus, the higher resistance to exhalation in such a nasal device can be suspended or delayed by the airflow resistor bypass. Suspension or delay (e.g., delay of onset) of the increase in resistance to exhalation may permit a subject to acclimate to wearing a nasal device that inhibits exhalation more than inhalation, particularly when the device is applied prior to sleeping.

In general, the nasal devices described herein are passive nasal devices, in which a resistance to exhalation is applied by a passive airflow resistor that does not require the application of additional airflow (e.g., from a source of pressurized gas, or the like) to increase the resistance to exhalation. These nasal devices generally include an opening configured to communicate with a nasal passage (e.g., nostril, nares, etc.), an airflow resistor in communication with the opening (wherein the airflow resistor is configured to increase the resistance to air exhaled through the opening more than air inhaled through the opening), and an airflow resistor bypass that can suspend the ability of the airflow resistor to increase the resistance nasal exhalation compared to nasal inhalation. The airflow resistor bypass allows the nasal device to ‘bypass’ the higher resistance to exhalation through the device due to the airflow resistor. Various examples of airflow resistor bypasses are described below.

A nasal devices including an airflow resistor and an airflow resistor bypass may be configured to manually suspend the increased resistance to exhalation (e.g., by manually activating the airflow resistor bypass), or they may be pre-set so that the airflow resistor bypass is active when the device is first applied to the subject. The airflow resistor bypass may also be configured to manually deactivate, allowing the airflow resistor to inhibit exhalation more then inhalation. In some variations the device may be configured to automatically inactivate the airflow resistor bypass and allow the airflow resistor to inhibit exhalation more than inhalation after some delay period. For example, the airflow resistor bypass may be configured to bypass the airflow resistor for a predetermined delay period after activation of the airflow resistor bypass. In some variations, the airflow resistor bypass may be manually disabled, allowing the airflow resistor to inhibit exhalation more than inhalation. One or more controls or triggers may be included as part of the nasal device and configured to allow inactivation or activation of the airflow resistor bypass.

For example, described herein are nasal respiratory devices including: an opening configured to communicate with a nasal passage, an airflow resistor in communication with the opening (wherein the airflow resistor is configured to increase the resistance to air exhaled through the opening more than air inhaled through the opening), and an airflow resistor bypass that is configured to suspend or reduce the increased resistance to exhalation applied by the airflow resistor, wherein the airflow resistor bypass is configured to be inactivated.

These nasal devices may also include a manual trigger for activating the airflow resistor bypass, wherein operating the trigger activates the airflow resistor bypass and decreases the resistance to exhalation through the nasal device. The trigger may be a button, switch, lever, tab, etc. (or any other appropriate control) on the nasal device.

The airflow resistor bypass may be configured to be manually inactivated by manipulating a control (e.g., a trigger, or the like). For example, the airflow resistor bypass may be configured to be automatically inactivated after a delay period. The delay period may be greater than 5 minutes, greater than 10 minutes, greater than 15 minutes, etc. The delay period may be less than 4 hours, less than 3 hours, less than 2 hours, less than 1 hour, or the like. Any appropriate delay period may be used, and the delay period may be fixed, or variable. In general, the delay period may be sufficiently long to allow a subject to fall asleep with the device, so that the airflow resistor becomes active after the patient falls asleep.

A nasal device may include an airflow resistor bypass that temporarily decreases the resistance to air exhaled through the device, bypassing the resistance applied by the airflow resistor to exhalation. In particular, nasal devices may include a holdfast configured to secure the respiratory device at least partly over or within a nasal passage. The holdfast may be an adhesive holdfast. Furthermore, these nasal devices may have any appropriate airflow resistor, including flap valve airflow resistors.

As mentioned, an airflow resistor bypass generally ‘bypasses’ the operation of the airflow resistor. In some variations, the airflow resistor bypass creates a transient decrease in the resistance to air flowing through the nasal device during exhalation by temporarily disabling or bypassing the airflow resistor. The airflow resistor bypass may decreases (or remove) the resistance to airflow through the nasal device while the airflow resistor bypass is activated. The airflow resistor bypass may be inactivated manually (e.g., by moving it away from the airflow resistor so that it no longer inhibits function of the airflow resistor, or by removing or reducing a bypass pathway), or automatically, after some delay period passes. In variations of the nasal device in which the airflow resistor bypass is automatically inactivated, the airflow resistor bypass is configured to bypass the airflow resistor for a delay period; the delay period may last for seconds, minutes, hours or days. The length of the delay period may depend upon the structure of the airflow resistor bypass and/or the materials forming the airflow resistor bypass or components of the airflow resistor bypass, as described in greater detail below. In some variations, the delay period is predetermined. The delay period may be adjustable or variable. The delay period for an airflow resistor bypass typically lasts longer than the event triggering activation of the airflow resistor bypass. For example an airflow resistor bypass can be activated when a subject wearing the device forcefully exhales through the device, or when the subject pushes a button or operates a control on the nasal device to activate the airflow resistor bypass, and remains active (e.g., decreasing the resistance to exhalation through the nasal device) for the duration of the delay period extending after the triggering event has ended.

Different variations of airflow resistor bypasses are described herein, any of which may be used with nasal devices having an airflow resistor configured to provide greater resistance to exhalation than to inhalation. An airflow resistor bypass that suspends the operation of the airflow resistor for a delay period may be referred to as a “delay bypass.” In some variations, an airflow resistor bypass includes a bypass channel forming a passageway through which air may pass during exhalation during a delay period, thereby bypassing the airflow resistor. A bypass channel can be regulated (e.g., opened/closed) by a bypass occluder, so that the bypass channel remains open during the delay period, but is closed (or substantially closed) thereafter. For example, an airflow resistor bypass may include a bypass channel that is located adjacent to the airflow resistor that can be covered by a bypass occluder (e.g., a flap). The bypass occluder acts as a timer. The bypass occluder (or a portion thereof, e.g, a hinge region) will eventually (e.g., after the delay period) return the bypass channel to the closed position, restoring the resistance to exhalation through the device from the airflow resistor. In some variations the bypass occluder is made (at least in part) of a material having a slow recovery from elastic deformation. Thus, the material can be displaced from an original shape configured to obstruct the bypass channel, and gradually return to the original shape to close the bypass channel.

In some variations, the airflow resistor bypass disengages the airflow resistor and prevents or reduces the resistance to exhalation for at least the delay period. Thus, an airflow resistor bypass may include a bypass displacer for displacing all or a portion of the airflow resistor during the delay period. For example, the airflow resistor bypass may include a bypass displacer configured as a bypass hinge that is connected to at least a portion of the airflow resistor. The bypass displacer can be activated to move the airflow resistor at least partially away from the passageway, permitting exhalation through the passageway that is unregulated by the airflow resistor. The airflow resistor bypass may move a valve portion (e.g., flap or flaps) of the airflow resistor out of the passageway. In some variations, the airflow resistor bypass acts by holding the valve of the airflow resistor open (or partially open) for the delay period. For example, the airflow resistor bypass may include a bypass displacer that holds the airflow resistor in an open configuration. In some variations, the airflow resistor bypass disables the airflow resistor in other ways. For example, the airflow resistor bypass may be configured to include a bypass displacer that prevents the valve limiter of an airflow resistor from holding the airflow resistor closed during exhalation. A bypass displacer may move a flap valve support(s) so that it does not engaged the flap valve in the closed position during exhalation. After the delay period, the bypass displacer disengages and the airflow resistor again provides an increased resistance to airflow during exhalation.

The bypass displacer may be an adhesive or other material that releasably secures all or a portion of the airflow resistor and releases it after the delay period. For example, a bypass displacer may be an adhesive that holds a flap of a flap-valve type airflow resistor in an open position until the adhesive releases the flap. The adhesive may be selected so that it releases after an appropriate delay. In some variations, an airflow resistor bypass may also be configured to expand the opening through which air may pass through the nasal device. For example, a nasal device may include a leak pathway that is typically open even during exhalation; an airflow resistor bypass may temporarily enlarge or increase the leak pathway.

A bypass displacer may also determine the delay period, and thus act as a bypass timer. For example, the bypass displacer may be a hinge or other structure that displaces the airflow resistor and then returns it to a preset position after the delay period. The delay period of the airflow resistor bypass may be any appropriate time period, from seconds to hours (or days). In some variations, the delay period is greater than 5 minutes, greater than 10 min, greater than 15 minutes, greater than 1 hour, greater than 2 hours, greater than 3 hours, greater than 4 hours, etc. The bypass period may be determined by a delay timer.

Any of the airflow resistor bypasses described herein may include a control or trigger for activating the airflow resistor bypass. For example, a trigger may comprises a push or pull tab. In some variations, the bypass occluder or bypass displacer is the trigger. Activating the airflow resistor bypass decreases the resistance to exhalation of the airflow resistor, starting the delay period, which continues even after the activation has ended. The resistance to exhalation is sustained by the airflow resistor bypass for the entire delay period (typically greater than one second). In some variations the airflow resistor bypass is triggered when a subject wearing the device exhales above some threshold force. In some variations the nasal devices are pre-activated, so that before the device is applied to a subject the airflow resistor bypass prevents the application of a high exhalation resistance by the airflow resistor. After being worn by the subject, the airflow resistor bypass may be manually deactivated (e.g., by manipulating a control or trigger), or it may automatically deactivate. For example, the airflow resistor bypass may include a moisture absorbing or swellable material that deactivates the airflow resistor bypass as it absorbs moisture from the respiring subject.

Also described herein are nasal respiratory devices configured to decrease resistance to exhalation through the device for a delay period. The devices include an opening (e.g., a passageway) configured to communicate with a nasal passage, an airflow resistor in communication with the opening (wherein the airflow resistor is configured to increase the resistance to air exhaled through the opening more than air inhaled through the opening), and an airflow resistor bypass including a bypass channel, where the airflow resistor bypass is configured to transiently decrease the resistance to air exhaled through the passageway for a delay period after activation of the airflow resistor bypass by opening the bypass channel through which air may be exhaled.

Also described herein are nasal respiratory devices configured to decrease resistance to exhalation through the device for a delay period that include an opening configured to communicate with a nasal passage, an airflow resistor in communication with the opening (wherein the airflow resistor is configured to increase the resistance to air exhaled through the opening more than air inhaled through the opening), and an airflow resistor bypass including a bypass displacer. The airflow resistor bypass is configured to transiently decrease the resistance to air exhaled through the opening for a delay period after activation of the airflow resistor bypass by engaging at least a portion of the airflow resistor with the bypass displacer.

Methods of suspending or deactivating the operation of a nasal device that is configured to inhibit exhalation more than inhalation are also described. In general, these methods may be applied to a subject wearing a passive nasal device that includes an airflow resistor that inhibits exhalation more than inhalation. These methods may include the steps of activating an airflow resistor bypass to suspend the application of increased expiratory resistance through the airflow resistor. Activation of the airflow resistor bypass may include opening a bypass pathway through the nasal device, moving the airflow resistor out communication with an opening through the device, or preventing the airflow resistor from closing during exhalation (or some combination of these).

Also described herein are methods of temporarily decreasing the resistance to exhalation through a nasal respiratory device, where the nasal respiratory device has an airflow resistor configured to be positioned in communication with a nasal passage so that the airflow resistor can increase the resistance to exhalation through the device more than inhalation through the device. The method of decreasing resistance through the nasal respiratory device includes the steps of: activating an airflow resistor bypass on the device, and decreasing the resistance to air exhaled through the device for a delay period after activation of the airflow resistor bypass. As described above, the airflow resistor bypass may suspend operation of the airflow resistor on the device. For example, the airflow resistor bypass may hold the airflow resistor open (preventing it from increasing the resistance to exhalation substantially), may move the airflow resistor so that it cannot substantially increase resistance to exhalation, or it may open or create a bypass pathway to circumvent the airflow resistor (e.g., by enlarging or creating a leak pathway).

Also described herein are methods of temporarily decreasing the resistance to exhalation through a nasal respiratory device that include the steps of applying a nasal device at least partially over the subject's nose without covering the subject's mouth, wherein the nasal device includes an opening configured to communicate with the subject's nasal passageway and an airflow resistor configured to inhibit exhalation through the nasal device more than inhalation through the nasal device, triggering activation of the airflow resistor bypass (or triggering a decrease in the resistance through the nasal device during exhalation) and decreasing the resistance to exhalation through the nasal device, wherein the decrease in resistance to exhalation is sustained for a delay period after triggering. The method may also include the step of restoring the resistance to exhalation through the nasal device.

As mentioned above, in some variations, the decrease in the resistance through the nasal device is triggered by activating an airflow resistor bypass on the device that interferes with the airflow resistor. For example, the airflow resistor bypass may move the airflow resistor out of way. In some variations, the resistance is decreased by engaging at least a portion of the airflow resistor with a bypass displacer, thereby temporarily disabling the airflow resistor. In some variations, the resistance is decreased by opening a bypass channel through which air may be exhaled.

Also described herein are methods of acclimating a subject to a nasal respiratory device. In some variations, the subject may be acclimated over a number of days, for example, by applying devices having increasing resistance to exhalation over consecutive days. These devices may also include airflow resistor bypasses. In some variations, the method of acclimating may be performed using a single device, or in a single session. For example, a method of acclimating may include the steps of applying a nasal device at least partially over the subject's nose without covering the subject's mouth (wherein the nasal device includes an opening configured to communicate with the subject's nasal passageway and an airflow resistor configured to inhibit exhalation through the nasal device more than inhalation through the nasal device), and gradually increasing the resistance to exhalation through the nasal device. The gradual increase may be achieved by gradually releasing an airflow resistor bypass, allowing the airflow resistor to inhibit exhalation more and more.

### INCORPORATION BY REFERENCE

All publications and patent applications mentioned in this specification are herein incorporated by reference in their entirety, as if each individual publication or patent application was specifically and individually indicated to be incorporated by reference in its entirety.

### BRIEF DESCRIPTION OF THE DRAWINGS

FIGS. 1A and 1B are a bottom and top perspective views, respectively, of one variation of a nasal device.

FIGS. 2A and 2B show one variation of a layered nasal device in a top view and an exploded perspective view, respectively.

FIG. 3A is a resistance profile for a nasal device, and FIG. 3B shows the effect of an airflow resistor bypass on the resistance profile of FIG. 3A.

FIGS. 4A and 4B are a nasal device including an airflow resistor bypass.

FIGS. 4C-4F show another variation of a nasal device including an airflow resistor bypass having an adhesive bypass displacer.

FIGS. 5A and 5B show another variation of an airflow resistor bypass.

FIG. 6A is a perspective view of a nasal device including an airflow resistor bypass. FIG. 6B show the nasal device from FIG. 6A in which the airflow resistor bypass is activated. FIGS. 6C-6F illustrate different variations of airflow resistor bypasses.

FIGS. 7A-7D illustrate another variation of an airflow resistor bypass.

FIG. 8A is a transparent perspective view of a portion of a nasal device including an airflow resistor bypass. FIG. 8B shows a segment of the airflow resistor bypass of FIG. 8A in an inactivated state.

FIG. 9A is transparent perspective view of a portion of a nasal device including an airflow resistor bypass. FIG. 9B shows a cross-section through the airflow resistor bypass of FIG. 9A.

FIG. 10 is a cross-section through another variation of a nasal device including an airflow resistor bypass.

FIG. 11A is a side cross-sectional view of another variation of an airflow resistor bypass. FIG. 11B is a top perspective view of the portion of the nasal device shown in FIG. 11A.

FIG. 12A is a perspective view of an airflow resistor bypass. FIG. 12B illustrates activation of the airflow resistor bypass of FIG. 12A when this airflow resistor bypass is part of a nasal device worn in communication with a subject's nasal passage.

FIG. 13A is a perspective view of a portion of a nasal device and an active airflow resistor bypass. FIG. 13B is a side view of the nasal device and airflow resistor bypass shown in FIG. 13A.

FIG. 14 shows an exemplary plot of resistance over time in a nasal device having an airflow resistor bypass.

FIGS. 15A and 15B show a bottom perspective and top views, respectively, of one variation of a nasal device having an airflow resistor bypass, including an activating button.

FIG. 15C shows a bottom view of another nasal device having an airflow resistor bypass.

FIGS. 16A and 16B illustrate perspective views of an airflow resistor bypass similar to that shown in FIGS. 15A and 15B. FIG. 16B is an exploded view.

FIGS. 17A and 17B are perspective, and cut-away perspective views, respectively, of another variation of an airflow resistor bypass.

FIG. 18 is an exploded view of a nasal device including an airflow resistor bypass.

FIGS. 19A and 19B are side and bottom perspective views, respectively, of another variation of an airflow resistor bypass, and FIG. 19C is an exploded view of the same airflow resistor bypass.

FIGS. 20A and 20B are bottom perspective and side views, respectively, or another variation of an airflow resistor bypass.

FIGS. 21A and 21B illustrate wearing and operation of a nasal device having an airflow resistor bypass with a bypass displacer that includes a delay adhesive.

FIGS. 22A and 22B illustrate another variation of an airflow resistor bypass in cross-sectional view.

FIGS. 23A and 23B show top and bottom views, respectively, of a nasal device including an airflow resistor bypass with a suction delay.

FIG. 23C is another variation of a nasal device including an airflow resistor bypass with a suction delay.

FIGS. 24A and 24B illustrate another variation of an airflow resistor bypass, in cross-section.

FIG. 24C is one example of a portion of an inflatable bypass displacer of an airflow resistor bypass.

FIGS. 24D and 24E illustrate operation of an airflow resistor bypass as described in FIGS. 24A-24C.

FIGS. 25A and 25B are side perspective views of another variation of an airflow resistor bypass.

FIGS. 25C and 25D are side and bottom perspective views, respectively, or a nasal device including the airflow resistor bypass illustrated in FIGS. 25A and 25B.

FIG. 26A is a perspective view of a portion of the airflow resistor bypass, including the bypass displacer, shown in FIGS. 25A-25D, and FIG. 26B is a section through a the airflow resistor bypass of FIGS. 25A-25D.

FIGS. 26C and 26D illustrate the airflow resistor bypass of FIGS. 25A-25D in the activated and inactivated state, respectively, in sectional views.

FIGS. 27A and 27B illustrate the operation of another variation of an airflow resistor bypass portion of a nasal device, in the inactivated and activated state, respectively.

### DETAILED DESCRIPTION OF THE INVENTION

Described herein are delayed resistance nasal devices. Delayed resistance nasal devices are nasal devices that have a higher resistance to exhalation than to inhalation during normal operation (e.g., when being worn by the subject, and particularly a sleeping subject), but these devices also include a bypass (e.g., airflow resistor bypass), so that the higher resistance to exhalation may be temporarily suspended or the onset delayed. While the airflow resistor bypass is active, the resistance to exhalation is lower than the resistance to exhalation during ‘normal’ operation, when the airflow resistor bypass is not active and the airflow resistor is inhibiting exhalation more than inhalation. In some variations, the resistance to exhalation during the period that the airflow resistor bypass is active is approximately the same as (or slightly more or less than) the resistance to inhalation. In general, the delayed resistance nasal devices described herein include an airflow resistor bypass that can be activated to bypass the effect of the airflow resistor of the nasal device, thereby decreasing the resistance to exhalation through the nasal device.

As described briefly above, an airflow resistor bypass may be manually activated after the nasal device is applied. An airflow resistor bypass may be pre-set so that it is active until the nasal device is worn by a subject for some time period, after which the airflow resistor bypass inactivates, allowing the airflow resistor to inhibit exhalation more than inhalation. Furthermore, an airflow resistor bypass may be configured to be inactivated manually or automatically. For example, an airflow resistor bypass may be configured to inactivate after some delay period (e.g., bypass delay period). Alternatively, it may be manually activated by operating a control such as button, switch, trigger, or the like. In some variations, the airflow resistor bypass may be inactivated manually or automatically (e.g., if not manually inactivated before the delay period ends).

Nasal devices having an airflow resistor bypass, including airflow resistor bypasses that are capable of temporarily suspending or decreasing the resistance to exhalation for some period of time (a “delay period”), are described in detail in the sections that follow. Methods of suspending (e.g., temporarily decreasing) the resistance to exhalation, as well as methods of using of nasal device capable of temporarily decreasing the resistance to exhalation, are also described. In addition, nasal devices that delay the onset of a high level of resistance to exhalation, and methods of using them to acclimate a subject to the operation of the nasal devices, are also described. Although the descriptions of the various devices and components of these nasal devices is divided into sections, any of the elements and components described in each of these sections may be incorporated or used with any of the elements and components described in any of the other sections.

As used in this specification, the singular forms “a,” “an,” and “the” include plural reference unless the context clearly dictates otherwise.

### Nasal Devices

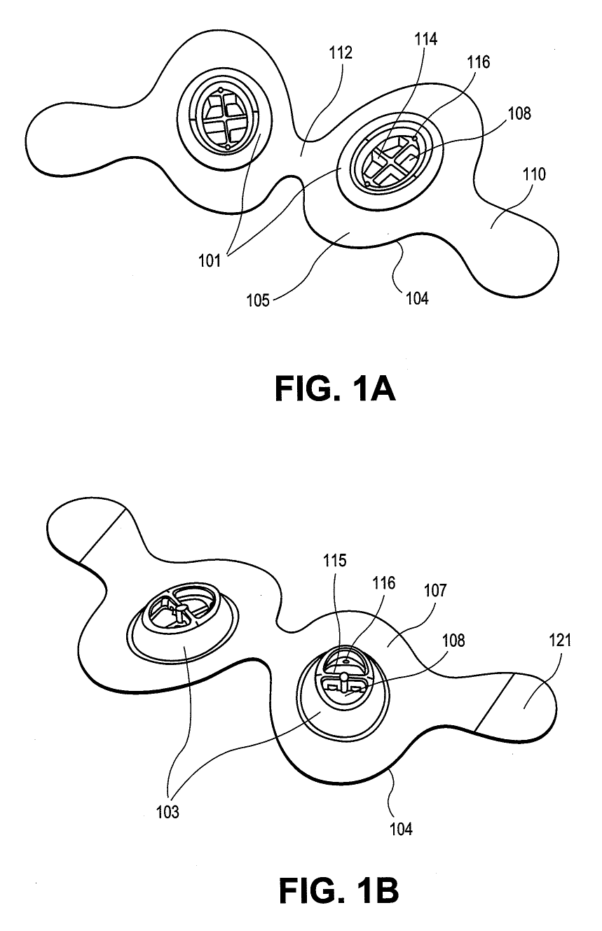

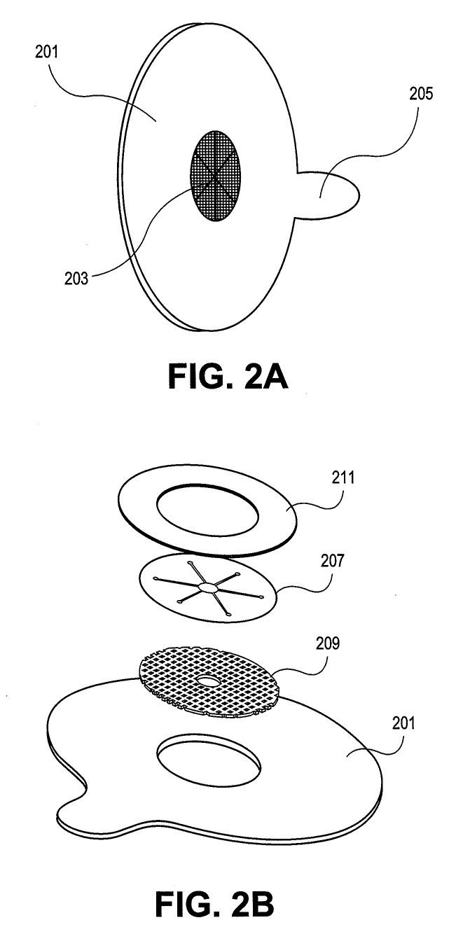

Any appropriate nasal device may be configured as a delayed resistance nasal device, particularly adhesive nasal devices, including those described in more detail in FIGS. 1A to 2B, below. The delayed resistance nasal devices described herein typically include a passageway (which may be just an opening) configured to communicate with a subject's nasal passage (or cavity), an airflow resistor in communication with the passageway, and an airflow resistor bypass. Nasal devices having flap valve airflow resistors may be particularly useful.

In general, a nasal device may be secured in communication with a subject's nose, and specifically with one or both of the subject's nasal cavities. A typical nasal device includes an airflow resistor that is configured to resist airflow in a first direction more than airflow in a second direction, and may also include an adhesive holdfast configured to secure the airflow resistor at least partially over, in, and/or across the subject's nose or nostril. The holdfast may include a biocompatible adhesive and a flexible region configured to conform to at least a portion of a subject's nose. The nasal devices described herein are predominantly adhesive nasal devices, however the systems and methods for packaging and dispensing nasal devices may be used with nasal devices that are not adhesive nasal devices.

Nasal devices may be worn by a subject to modify the airflow thorough one or (more typically) both nostrils. One or more nasal devices may be secured over both of the subject's nostrils so that airflow through the nostrils passes primarily or exclusively through the nasal device(s). Adhesive nasal devices are removably secured over, partly over, and/or at least partly within the subject's nostrils by an adhesive. The nasal devices described herein may be completely flexible, or partially rigid, or completely rigid. For example, the devices described herein may include an adhesive holdfast region that is at least partially flexible, and an airflow resistor. The airflow resistor may be flexible, or rigid. In some variations, the devices described herein also include one or more alignment guides for helping a subject to orient the device when securing it over the subject's nose. The adhesive nasal devices described herein may be composed of layers. Nasal devices composed of layers (which may also be referred to as layered nasal devices) may be completely or partially flexible, as previously mentioned. For example, a layered nasal device may include an airflow resistor configured to resist airflow in a first direction more than airflow in a second direction and an adhesive holdfast layer. In some variations, the airflow resistor may be a flap valve layer adjacent to a flap valve limiting layer, and may include an adhesive holdfast layer comprising an opening across which the airflow resistor is operably secured. The airflow resistor may be disposed substantially in the plane of the adhesive holdfast layer. The adhesive holdfast layer may be made of a flexible substrate that includes a biocompatible adhesive.

Nasal respiratory devices, including adhesive respiratory devices, may be used to regulate a subject's respiration. For example, a nasal device may create positive end expiratory pressure (“PEEP”) or expiratory positive airway pressure (“EPAP”) during respiration in a subject wearing the device. The nasal devices and methods described herein may be useful to treat a variety of medical conditions, and may also be useful for non-therapeutic purposes. For example, a nasal respiratory device may be used to treat sleep disordered breathing or snoring. The systems, devices and methods described herein are not limited to the particular nasal device embodiments described. Variations of the embodiments described may be made and still fall within the scope of the disclosure.

As used herein, a nasal device may be configured to fit across, partly across, at least partly within, in, over and/or around a single nostril (e.g., a “single-nostril nasal device”), or across, in, over, and/or around both nostrils (“whole-nose nasal device”). Any of the features described for single-nostril nasal devices may be used with whole-nose nasal devices, and vice-versa. In some variations, a nasal device is formed from two single-nostril nasal devices that are connected to form a unitary adhesive nasal device that can be applied to the subject's nose. Single-nostril nasal devices may be connected by a bridge (or bridge region, which may also be referred to as a connector). The bridge may be movable (e.g., flexible), so that the adhesive nasal device may be adjusted to fit a variety of physiognomies. The bridge may be integral to the nasal devices. In some variations, single-nostril nasal devices are used that are not connected by a bridge, but each include an adhesive region, so that (when worn by a user) the adhesive holdfast regions may overlap on the subject's nose.

An airflow resistor bypass is typically included as part of the nasal device. Airflow resistor bypasses may include a bypass channel that is open when the airflow resistor bypass is active, and/or they may include a bypass displacer that temporarily removes or inactivates all or a portion of the airflow resistor. In some variations a “bypass timer” is included that determines (at least in part) the duration of the delay period of the airflow resistor bypass. In some variations the bypass timer is a portion of the airflow resistor bypass whose structural, mechanical or chemical properties determine the delay period when the airflow resistor bypass is activated. A description (and examples) of various airflow resistor bypasses are provided below, following the description of the generic nasal devices shown in FIGS. 1A-2B.

One variation of a nasal device that may include an airflow resistor bypass is a layered nasal device, formed of two or more layers. For example, a layered nasal device may include an adhesive holdfast layer and an airflow resistor layer. These layers may themselves be composed of separate layers, and these layers may be separated by other layers, or they may be adjacent. For example, the adhesive holdfast layer may be formed of layers (optionally: a substrate layer, a protective covering layer, an adhesive layer, etc), and thus may be referred to as a layered adhesive holdfast. Similarly, the airflow resistor may be formed of multiple layers (optionally: a flap valve layer, a valve limiter layer, etc.), and thus may be referred to as a layered airflow resistor. In some variations, the layered adhesive holdfast and the layered airflow resistor share one or more layers. For example, the flap valves layer and the adhesive substrate layer may be the same layer, in which the leaflets of the flap valve layer are cut from the substrate layer material. As used herein, a “layer” may be a structure having a generally planar geometry (e.g., flat), although it may have a thickness, which may be uniform or non-uniform in section. As mentioned briefly above, the support backing may be formed of one of the layers of a layered nasal device, such as the adhesive substrate layer.

In some variations, a nasal device has a body region including a passageway configured to be placed in communication with a subject's nasal passage. The body region may be a stiff or flexible body region, and may secure an airflow resistor therein. In some variations, the body region is at least partially surrounded by a holdfast (i.e., a planar adhesive holdfast). The body region may be modular, meaning that it is formed of two or more component sections that are joined together. Examples of such nasal devices can be found in U.S. Ser. No. 11/811,339, filed on Jun. 7, 2007, herein incorporated by reference in its entirety.

In some variations, the adhesive nasal device includes a support frame. The support frame may provide structural support to all or a portion of the nasal device, such as the flexible adhesive portion. For example, the support frame may support the adhesive holdfast portion of the device and be completely or partially removable after the device has been applied to the subject. In some variations, the support frame remains on the nasal device after application. In some variations, the support frame is a support frame layer.

An adhesive nasal device may also include a tab or handle configured to be grasped by a subject applying the device. In some variations, this tab or handle is formed of a region of the layered adhesive holdfast.

The various components of the device may be made of any appropriate materials, as described in greater detail below. For example, some device components (e.g., an alignment guide, a body region) may be made of medical grade plastic, such as Acrylonitrile Butadiene Styrene (ABS), polypropylene, polyethylene, polycarbonate, polyurethane or polyetheretherketone. The airflow resistor may be a flap valve and the flap may be made of silicone or thermoplastic urethane. The adhesive holdfast may include an adhesive substrate made of silicone, polyurethane or polyethylene. Examples of biocompatible adhesive on the adhesive holdfast may include hydrocolloids or acrylics. These lists of materials are not exclusive, and other (or alternative) materials may be used.

In some versions, the nasal device further comprises an active agent. In some versions, this active agent is a drug (e.g., a medicament). In some versions, this active agent comprises an odorant, such as a fragrance. In some versions, the active agent comprises menthol, eucalyptus oil, and/or phenol. In other versions, the nasal device may be used with other pulmonary or medical devices that can administer medication or other medical treatment, including, but not limited to, inhalers and nebulizers.

A nasal device may include a filter. This filter may be a movable filter, such as a filter that filters air flowing through the passageway in one direction more than another direction (e.g., the device may filter during inhalation but not exhalation).

As mentioned, the adhesive nasal devices described herein typically include a holdfast region (or layer) and at least one airflow resistor. As will be apparent from the figures, many of these nasal devices may be removable and insertable by a user without special tools. In some variations, a subject may use an applicator to apply the device (e.g., to help align it). FIGS. 1A through 2B illustrate different exemplary nasal devices.

FIGS. 1A and 1B show perspective views of one exemplary variation of an adhesive nasal device that may be configured as a delayed resistance nasal device and may include an airflow resistor bypass (a delayed bypass is not visible in these figures). FIG. 1A shows a front perspective view of an adhesive nasal device, looking at the “outer” side of the device, which is the side facing away from the subject's nose when the device is worn. The device shown in FIG. 1A includes two single-nostril rim bodies 101 and a single adhesive holdfast 104. A nasal device may be configured to communicate with a single nostril (a single-nostril nasal device), or it may be configured to communicate with both of a subject's nostrils (a double-nostril nasal device as shown here).

The holdfast 104 (which adhesively secures the device to the subject) is shown as a layered structure including a backing or adhesive substrate 105. This backing may act as a substrate for an adhesive material, or it may itself be adhesive. The holdfast 104 may have different regions, including two peri-nasal regions surrounding the rim bodies 101. Each rim body has at least one passageway 108 for airflow therethrough. The adhesive holdfast also includes two tabs or grip regions 110 that may make the device easier to grasp, apply, and remove. A bridge region 112 is also shown. In this example, the bridge region is part of the adhesive holdfast (e.g., is formed by the same substrate of the adhesive holdfast) and connects the peri-nasal regions. Although the tab and bridge regions are shown as being formed as part of (integral with) the holdfast material, these regions may also be formed separately, and may be made of different materials.

The rim body regions 101 shown in the exemplary device of FIG. 1A include outer rim body regions which each encompass a passageway 108. These first (e.g., outer) rim body regions may mate with second (e.g., inner) rim body regions to form the rim body region(s) of the device that each include a passageway 108. This passageway is interrupted by crossing support members 114 (e.g., cross-beams or cross-struts) that may partly support or restrict movement of the airflow restrictor. In addition, each rim body region 101 includes two leak pathways 116, through which air may pass even when the passageway through the device is otherwise blocked by the airflow resistors. The leak pathways 116 are shown here as small openings at the narrow ends of the oval-shaped outer rim body region. The rim body region may also be referred to as ‘rim’ or ‘scaffold’ regions of the device.

FIG. 1B shows a back perspective view of the opposite side of the adhesive nasal device shown in FIG. 1A, the “inner side” of the device. The inner side of the device faces the subject, and a portion of this side of the device may contact the subject. This side of the device, and particularly the adhesive holdfast of the device, includes an adhesive (which may be covered by a protective cover 107) forming part of the holdfast 104. In some variations, the entire skin-facing side of the holdfast 104 includes an adhesive on the surface, although in some variations, only a portion of this region includes adhesive. The adhesive may be a distinct layer of the holdfast (e.g., it may be layered on top of an adhesive substrate), or it may be an integral part of the holdfast (e.g., the adhesive substrate may be made of an adhesive material). In some variations an adhesive may be separately added to the device (e.g., the holdfast region) before use. The adhesive material may be covered by a removable protective cover or liner 107, to prevent the adhesive from sticking to surfaces until after the liner is removed. In FIG. 1B, the protective cover 107 covers the entire skin-facing surface of the holdfast. The device may be applied by first removing the liner. For example, the liner may be peeled off, to expose the adhesive. In some variations, the liner protecting the adhesive may be partially removed. For example, the tab region 121 of the device may include a separate (or additional) liner that remains over the tab region when other liner regions are removed. This may allow the device to be held by the tab region without having it adhere to the skin. After removing the cover, or a part of the cover, the device may be positioned and adhered to the subject's skin around the nasal cavity, so that the passageways through the rim body are aligned with the openings of the subject's nasal cavities. In some variations, an additional adhesive cover region (e.g., the protective cover region over the tabs 121) can then be removed to secure the device to the rest of the subject's nose. The adhesive cover may include a fold (or crimp, crease, lip, or the like) that helps to remove the protective cover from the adhesive.

The second, or inner, rim body region 103 shown in the exemplary device of FIG. 1B is shaped with an inwardly-tapering edge, so that it may fit at least slightly within the opening of the subject's nostril when a subject wears the device. The inner rim body includes one or more passageways 108 that correspond with the passageways 108 shown in FIG. 1A. Similarly, the leak pathways pass completely through the rim body (both inner and outer bodies). The tapering external walls of the inner rim body region(s) shown in FIG. 1B are shown as smooth, and may also include an additional material (e.g., an auxiliary holdfast material) for securing them in the subject's nostrils, or for cushioning them to prevent injury or discomfort. These surfaces may also be more or less angled, in order to facilitate comfort when the adhesive nasal device is worn in the subject's nose. A cross bar (hinge region 115) may also be provided as part of the inner rim body. The inner rim body 103 may extend some distance above the peri-nasal annular region of the holdfast, as shown in FIG. 1B. This distance may be sufficient to prevent any portion of the airflow resistor (e.g., a flap portion of a flap valve) from extending out of the device and into the nasal cavity where it might contact body tissues.

All of the nasal devices described herein also include an airflow resistor, which is located in one or more passageways formed through the device. In FIGS. 1A and 1B, the airflow resistor is a flap valve, and includes cross bars that support the flap valve (and can prevent it from opening during exhalation). In general, the airflow resistor opesn in one direction (during inhalation) and is closed during exhalation. The flap may be made of silicone. In the device shown in FIGS. 1A and 1B, the flap can be secured between the inner and outer rim bodies.

As described in greater detail below, an airflow resistor bypass may be incorporated into any portion of the nasal device, including the rim body region(s) 101, the leak pathways, the valve support members 114, etc. For example, the airflow resistor bypass may include a bypass channel through the rim body that can be opened when the airflow resistor bypass is activated. For example, the airflow resistor bypass may controllably open a new leak pathway or widen an existing leak pathway to at least partially bypass the airflow resistor and thereby temporarily reduce the resistance to exhalation. In some variations, the airflow resistor bypass includes a bypass displacer attached to a part of the nasal device (e.g., the rim) that can be temporarily moved to disable the airflow resistor so that it is held at least partially open. Bypass displacers will be described and illustrated below.

FIG. 2A is a top view of another example of a nasal device. The nasal device shown in FIGS. 2A-2B is a layered nasal device that includes a holdfast layer 201 and an airflow resistor 203. The reverse side of the device shown in FIG. 2A includes an adhesive material (not shown) that may be covered by a protective covering. The protective covering (which may also be referred to as a protective liner) can be removed to expose the adhesive before application of the device. Thus, the holdfast layer of the device secures it to the subject. This holdfast layer may itself be layered, and may include an adhesive substrate (e.g., a backing layer). For example, the adhesive substrate may be a foam backing. This backing may act as a substrate for an adhesive material. In some variations, the adhesive substrate is itself adhesive. The holdfast layer 201 may have different regions, including a peri-nasal regions surrounding a passageway (though which air may flow), and a tab 205 or grip region forming a tab that may make the device easier to grasp, apply and remove. Other regions may include regions of more aggressive and less aggressive adhesive (e.g., more or less adhesive material), or regions of hydrogel material (including adhesive hydrogels) to help prevent irritation from repeated or extended use. Although the tab is shown as part of (integral with) the holdfast material, this region may also be formed separately, and may be made of different materials.

FIG. 2B shows an exploded view of the device of FIG. 2A. This exploded perspective view illustrates the layers of the device, including the adhesive holdfast 201 (which may itself be layered), two layers forming the airflow resistor, including the flap valve 207 and flap valve limiter 209, and an adhesive ring 211 that may help attach the flap valve and flap valve limiter to the adhesive holdfast.

An adhesive holdfast for a nasal device may comprise any appropriate material. For example, the adhesive substrate may be a biocompatible material such as silicone, polyethylene, or polyethylene foam. Other appropriate biocompatible materials may include some of the materials previously described, such as biocompatible polymers and/or elastomers. Suitable biocompatible polymers may include materials such as: a homopolymer and copolymers of vinyl acetate (such as ethylene vinyl acetate copolymer and polyvinylchloride copolymers), a homopolymer and copolymers of acrylates (such as polypropylene, polymethylmethacrylate, polyethylmethacrylate, polymethacrylate, ethylene glycol dimethacrylate, ethylene dimethacrylate and hydroxymethyl methacrylate, and the like), polyvinylpyrrolidone, 2-pyrrolidone, polyacrylonitrile butadiene, polyamides, fluoropolymers (such as polytetrafluoroethylene and polyvinyl fluoride), a homopolymer and copolymers of styrene acrylonitrile, cellulose acetate, a homopolymer and copolymers of acrylonitrile butadiene styrene, polymethylpentene, polysulfones polyimides, polyisobutylene, polymethylstyrene and other similar compounds known to those skilled in the art. Structurally, the substrate may be a film, foil, woven, non-woven, foam, or tissue material (e.g., poluelofin non-woven materials, polyurethane woven materials, polyethylene foams, polyurethane foams, polyurethane film, etc.).

In variations in which an adhesive is applied to the substrate, the adhesive may comprise a medical grade adhesive such as a hydrocolloid or an acrylic. Medical grade adhesives may include foamed adhesives, acrylic co-polymer adhesives, porous acrylics, synthetic rubber-based adhesives, silicone adhesive formulations (e.g., silicone gel adhesive), and absorbent hydrocolloids and hydrogels.

An airflow resistor bypass may also be incorporated into any portion of a layered nasal device such as that illustrated in FIGS. 2A and 2B, including the holdfast layer, the airflow resistor (e.g., flap valve 207) or limiter (e.g., flap valve limiter 209). For example, a flap valve limiter may be configured as an airflow resistor bypass. Examples of airflow resistor bypasses that may be included with nasal devices, including the nasal devices illustrated in FIGS. 1A-2B are described below.

### Airflow Resistor Bypass

An airflow resistor bypass suspends the effect of an airflow resistor. For example, an airflow resistor bypass may temporarily and controllably decrease the resistance of airflow through a nasal device in at least one direction (typically the direction of expiratory airflow) from a normal operating (or baseline) resistance to a lower (or bypass) resistance. An airflow resistor bypass may decrease the baseline resistance to exhalation, Re, to a lower bypass resistance to exhalation, rₑ, so that rₑ<Rₑ. The airflow resistor bypass may maintain this lower resistance (rₑ) until it is inactivated. In some variations, the airflow resistor bypass maintains the lower resistance (rₑ) for a delay period of duration t_{d}. The lower resistance (rₑ) may be constant or variable. The baseline resistance to exhalation, Rₑ, is typically higher than the baseline resistance to inhalation, Rᵢ, (Rᵢ<Rₑ) and often Rᵢ is much lower then Rₑ (Rᵢ<<Rₑ).

In general, an airflow resistor bypass may be any structure that temporarily and controllably bypasses the effect of the airflow resistor, for example, by creating a bypass channel for air during at least the expiratory portion of respiration when the a nasal device with an airflow resistor bypass is worn and activated. In some variations, the airflow resistor bypass is a bypass displacer which disrupts all or a portion of the airflow resistor to decrease its resistance, including disrupting the airflow resistor by restricting movement of a portion of the airflow resistor, or by displacing all or a portion of the airflow resistor. The airflow resistor bypass may also include a structure forming a bypass channel that is a passageway that is open during activation of the airflow resistor bypass and circumvents the airflow resistor.

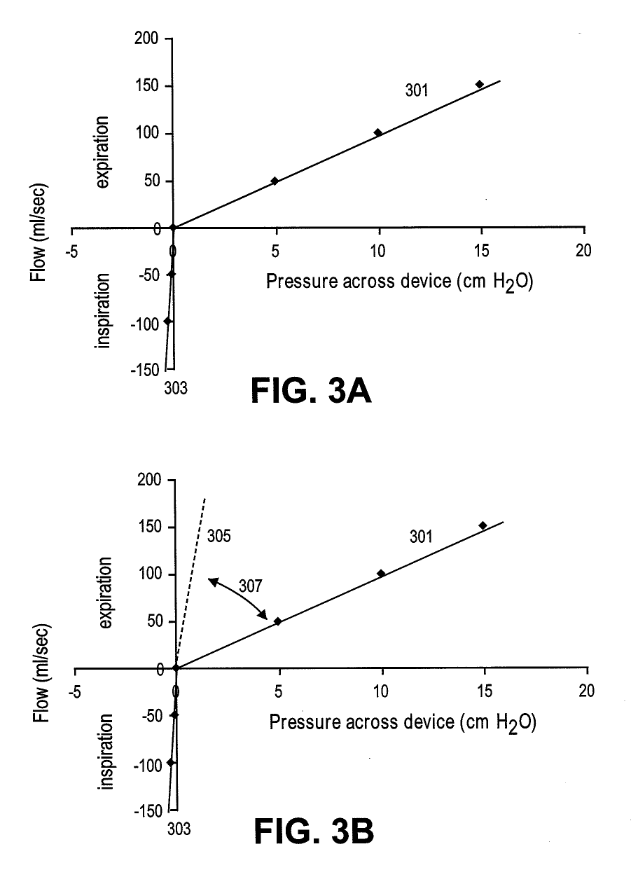

An airflow resistor bypass may decrease the resistance to exhalation in a nasal device by providing a “bypass route” for airflow by disabling or at least partially circumventing the airflow resistor. FIGS. 3A and 3B graphically illustrate the effect of an airflow resistor bypass on the flow at different pressures across a nasal device during inhalation and exhalation. FIG. 3A shows a hypothetical resistance profile for a nasal device in normal operation, having a greater resistance to airflow during exhalation 301 (shown here as positive flow) compared to inhalation 303 (shown as negative flow). The relationship between airflow and pressure across the device is simplified. During inhalation, a small change in the pressure across the device results in a large change in the flow. The linear relationship shown for the inhalation line 303 therefore has a very steep slope, corresponding to a small resistance. In contrast, during exhalation 301 the flow rate does not change as much in response to a change in pressure from the lungs, and thus the exhalation line has a much flatter slope, reflecting a larger resistance.

In FIG. 3B an airflow resistor bypass has been activated during inhalation, reducing the resistance to exhalation while the airflow resistor bypass is active. This is reflected in FIG. 3B as dashed line 305. During the period when the airflow resistor bypass is active, the resistance remains low for exhalation. Once this period has ended, the airflow resistor bypass becomes inactive, and the resistance to exhalation returns to the baseline level 301. The resistance to exhalation may be as low (or lower) than the resistance to inhalation (rₑ<Rᵢ), or it may still be slightly higher (rₑ>Rᵢ). Furthermore, the transition between the baseline resistance 303 and the lower resistance during activation of the airflow resistor bypass 305 may be quick or gradual (e.g., shifting from the higher resistance slope 301 to the lower resistance slope 305 as indicated by arrow 307). In FIGS. 3A and 3B, the resistance to inhalation is not substantially changed by the operation of the airflow resistor bypass. In some variations the resistance to inhalation may be changed by activation of the airflow resistor bypass. For example, the resistance to inhalation may be slightly decreased or slightly increased when the airflow resistor bypass is active.

An airflow resistor bypass may temporarily decrease the resistance to exhalation by creating a bypass channel or pathway (e.g., circumventing the airflow resistor), by using a bypass displacer to disable or modifying the airflow resistor (e.g., holding it open or partially open during exhalation), or by some combination of these two. An airflow resistor bypass may include one or more bypass channels and/or bypass displacers.

A bypass channel is generally a metered passage or opening through which airflow may pass to circumvent the airflow resistor. The bypass channel is metered so that it remains open and decreases resistance to exhalation for the duration of the delay period after activation of the airflow resistor bypass. For example, a bypass channel may be a passage through the nasal device adjacent to the airflow resistor that includes a compressible/slowly expandable material (e.g., any of the slow-recovering elastic deformation materials and/or memory materials described below) that at least partially occlude the bypass channel in the relaxed or uncompressed state. Compressing the material opens or exposes the bypass channel so that air may flow through the bypass channel. Eventually the occluding material expands back to at least partially block the bypass channel until the normal (baseline) operation of the airflow resistor is restored. The occluding material acts as a bypass timer because the duration of the delay period for the airflow resistor bypass maybe determined by the material and structural properties of the occluding material. Other examples of bypass channels are described below.

A bypass displacer is generally a structure or material that prevents the airflow resistor from increasing the resistance to airflow during exhalation. For example a bypass displacer may be a hinge, arm, beam, slider, or other appropriate structure that moves the valve limiter away from the valve, allowing the valve to open during inhalation and exhalation. In some variations the bypass displacer is a material or structure that prevents complete closure of the airflow resistor during exhalation. In some variations the bypass displacer is a post, tab, or other projection that holds the airflow resistor at least partially open during exhalation. For example, a bypass displacer may include an adhesive or sticky surface that releasably holds a portion of the airflow resistor open, even during exhalation. For example, when the airflow resistor is a flap valve, the bypass displacer may hold one or more flaps of the flap valve open during exhalation. In some variations, the bypass displacer disrupts the airflow resistor by blocking it at least partially open. In other variations, the bypass displacer disrupts the airflow resistor by moving the airflow resistor (e.g., flap valve) out of the passageway.

In operation, the airflow resistor bypass may be configured to be either multi-use or single-use. A single-use airflow resistor bypass may be configured to delay the onset of the higher baseline level of resistance during exhalation when the nasal device is first worn by the user. For example, a nasal device may be configured so that the airflow resistor bypass is activated even before (or upon) application of the device in communication with the subject's nose. Initially delaying the onset of the higher resistance to exhalation may allow a user to more readily acclimate to the device, particularly when the device is worn before sleeping. As mentioned above, in some variations the airflow resistor bypass gradually increases the bypass resistance to exhalation (from rₑ) to the baseline resistance to exhalation (Rₑ) during a delay period (t_{d}). In some examples, the airflow resistor bypass maintains the delay resistance at rₑ during most of the delay period. A single-use airflow resistor bypass may include an activator or trigger (e.g., a button, tab, etc.), or it may be automatically activated upon application in communication with a subject's nasal passage. For example, a nasal device may be configured so that the airflow resistor bypass is activated before the device is worn, but application of the device triggers the start of the delay period (e.g., body heat or pressure may trigger the start of the delay period, t). In other variations, the airflow resistor bypass includes a trigger that can be manually activated either before or after the device is worn by the user. The airflow resistor bypass trigger activates the airflow resistor bypass, and may start the delay period in variations that automatically inactivate.

In some variations an airflow resistor bypass device may be activated or triggered multiple times, each time suspending or decrease the inhibition of exhalation by the airflow resistor. Thus, an airflow resistor bypass may include a trigger or activator. Any appropriate trigger may be used, including a switch, tab, dial, button, squeeze trigger, etc. The airflow resistor bypass trigger may act as a “snooze” control that temporarily reduces or suspends the resistance to exhalation.

As described above, the airflow resistor bypass may also be deactivated manually. In some variations a control (e.g., trigger, button, etc.) may be used to inactivate the airflow resistor bypass. In some variations the same trigger used to activate the airflow resistor bypass may be used to inactivate it.

The airflow resistor bypass devices described herein may effectively decrease the resistance to exhalation for a delay period. The delay period may extend for seconds, minutes, or hours, depending upon the configuration of the airflow resistor bypass. Of course, in some variations, the delay period is manually determined by the user or a third party, who can operate the manual inactivation. In general the delay period extends longer than the time required to trigger the airflow resistor bypass, and may be sufficiently long enough to allow a subject wearing the device to fall assleep. For example, the delay period may begin after the activation of the airflow resistor bypass, during which time the resistance to exhalation is sustained for the duration of the delay period.

In some variations the delay period td lasts for minutes (e.g., between 3 and 60 minutes). For example, the delay period may be approximately 5 minutes, approximately 10 minutes, approximately 15 minutes, approximately 20 minutes, approximately 30 minutes, etc. The duration of the delay period may be constant (e.g., a predetermined time period) or it may be variable. For example, the duration of the delay period may be determined by the force applied to activate the airflow resistor bypass. In some variations, the length of the delay period is determined by the structure and/or material properties of all or a portion of the airflow resistor bypass. For example, if the airflow resistor bypass includes a bypass displacer made of a compressible/slow-expanding foam, the delay may be determined by the shape of the bypass displacer and its material properties. In some variations a more precise bypass timer may be used. For example, an electronic timer may be used to actively control the airflow resistor bypass. Thus, a nasal device may include electronic circuitry configured to control the airflow resistor bypass and thereby the resistance through the device during the delay period.

Described below are various examples of nasal devices including airflow resistor bypasses. These examples illustrate some of the operating principles of airflow resistor bypasses and nasal devices including airflow resistor bypasses.

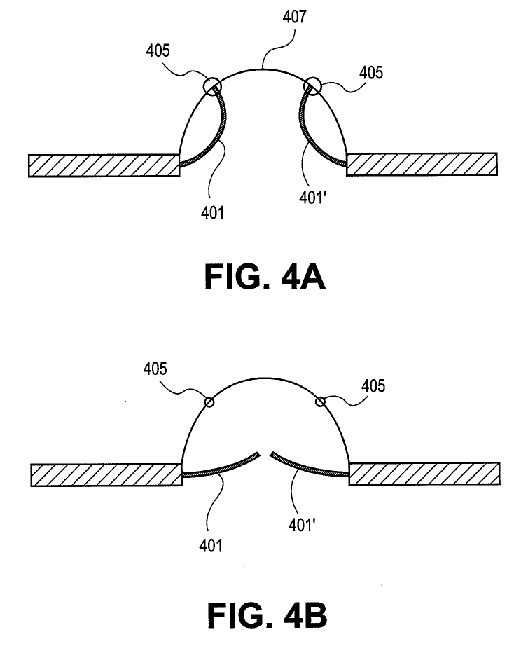

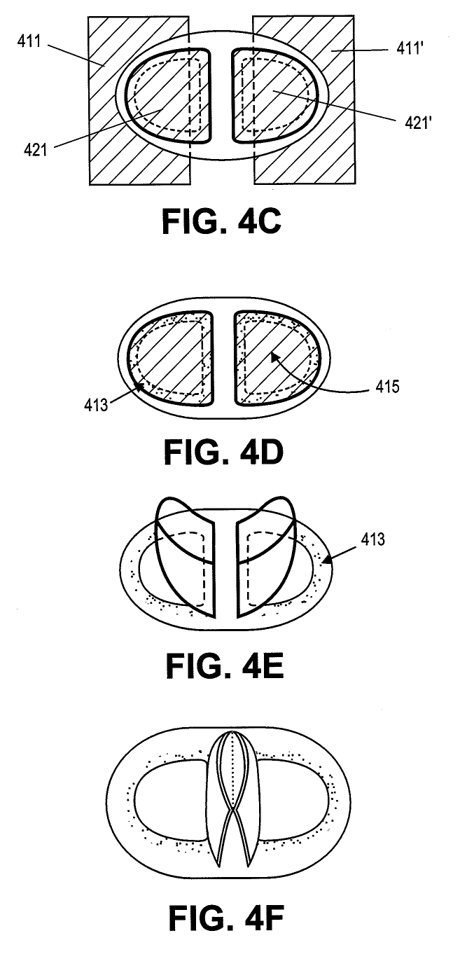

FIGS. 4A and 4B show a cross-section through a nasal device including an airflow resistor bypass having a bypass displacer. The airflow resistor in FIG. 4A is a flap valve having two flaps 401, 401′. The airflow resistor displacer in this example includes an adhesive material 405 that is located on the side of the device that will face the subject's nasal cavity (e.g., the proximal side). The adhesive can be configured so that it adheres to the flap valves 401, 401′, holding them in the open position, as shown in FIG. 4A. The adhesive may be selected and/or positioned so that it will only temporarily hold the flaps, since the flaps can be biased in the closed position, effectively “pulling” against the adhesive 405. In addition, exhalation through the passageway when the flaps are in the open position will also tend to push against the flaps, weakening the adhesive contact between the flaps and the airflow resistor displacer. Eventually (after the delay period), the flaps will release from the airflow resistor displacer, and the normal (or baseline) operation of the airflow resistor will increase the resistance to exhalation as the flaps close during exhalation.

The adhesive forming the bypass displacer(s) in FIGS. 4A and 4B are located on a bridge 407 that positions the adhesive surfaces so that they can contact the flap valves in the open position. In some variations the bridge includes the adhesive material, which can be applied to the bridge, or the bridge can be made of an adhesive material. In some variations, the adhesive material is located on the flap valves. In the cross-section shown in FIG. 4A and 4B, the adhesive forming the bypass displacer may be a ring or surface of adhesive, or a number of discrete regions of adhesive. For example, the entire inner surface (facing the valves) of the bridge may be coated with adhesive, or just a portion of the surface. In some variations a separate structure (e.g., bridge 407) is not necessary, and the adhesive forming the bypass displacer(s) are present on other components of the device, such as the adhesive holdfast.

The airflow resistor bypass shown in FIGS. 4A and 4B may be configured as a single-use airflow resistor bypass, or as a multi-use airflow resistor bypass. For example, the bypass displacer may include an adhesive that loses adhesion gradually after exposure to the moisture in the air passing from the nasal passages. If the nasal device is initially configured so that the airflow resistor (flap valves 401, 401′) is disabled by the airflow resistor bypass when the device is first worn by the user, the airflow resistor bypass will release the airflow resistor after the delay period during which respiration weakens the adhesive sufficiently to release the flap valves. In some variations the bypass displacer is a wax, or other material that is similarly temperature or moisture sensitive so that it releases some time after the device is worn by the subject.

The airflow resistor bypass shown in FIGS. 4A and 4B may be configured as a multi-use airflow resistor bypass. For example, the adhesive may repeatedly secure and release the flap valve(s). Activation of the airflow resistor bypass can be triggered manually. In one variation, the airflow resistor bypass is triggered by a forceful inhalation that results in the flap valves opening sufficiently (and/or for sufficient time) to contact and engage the bypass displacer adhesive 405. In one variation, a separate activator (e.g., a tool that inserts into the passageway of the nasal device) can be used to engage the airflow resistor bypass with the flap valves. For example, an activator may include a tab or post that inserts (e.g. past any flap valve restrictor) into the passageway and pushes the flap(s) of the airflow resistor against the adhesive of the airflow resistor bypass (the bypass displacer), activating it.

FIGS. 4C-4F show another variation of a nasal device including an airflow resistor bypass having an adhesive bypass displacer. FIG. 4C shows the side of the nasal device facing away from the user when the device is worn (e.g., the distal end of the nasal device). The nasal device has two flap valve limiters 421, 421′ on the distal side that prevent the flap valves from opening during exhalation. In this example, the airflow resistor bypass is configured to move the flap valve limiters away from the flap valves, and thereby permit the flap valves to open distally during exhalation, rather than closing and increasing the resistance to airflow during exhalation. In FIG. 4C the nasal device includes two protective covers 411, 411′ that cover the flap valve limiters and the underlying region until removed. Removing the protective covers exposes the bypass displacer adhesives 413, 415. This example uses two adhesives. A first adhesive 413 releasably secures the flap valve limiters 421, 421′ against the opening of the passageway through the device so that the flap valve limiter can prevent the flap valve from opening too far. The second adhesive 415 is located on the proximal side of the flap valve limiters (or at least one of the limiters), and can releasably hold the flap valve limiters in the open position, as illustrated in FIG. 4F.

In operation, the nasal device is first applied to a subject's nasal area, and the protective covers 411, 411′ are removed. The airflow resistor bypass can be activated. For example, the airflow resistor bypass in this example can be activated by an intense exhalation that is sufficient to cause the release of the flap valve limiters 421, 421′ from the first adhesive 413 so that the flap valve limiters can be moved away from the flap valves until they touch each other, and engage the second adhesive 415, as shown in FIGS. 4E-4F. After a delay period, the second adhesive releases and the flap valve limiter returns against the opening of the passage where it is secured in position by the adhesive (e.g., as in FIG. 4D).

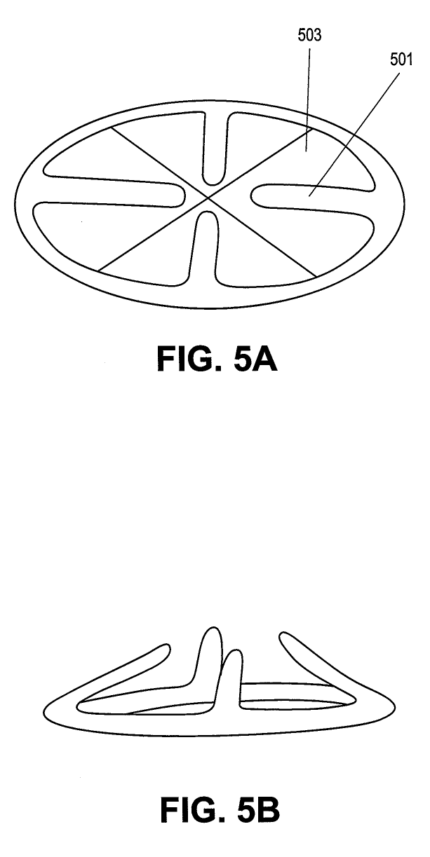

FIGS. 5A and 5B show a portion of a nasal device including another variation of an airflow resistor bypass. In this variation, the flap valve limiters 501 also act as the bypass displacers. In normal operation the flap valve limiters 501 prevent the leaflets of the flap valve 503 from opening in the distal direction (the direction of exhalation), thereby increasing the resistance to exhalation compared to inhalation. In this example, however, the flap valve limiters are configured so that sufficient force applied to the flap valve limiter allows it to temporarily move distally, permitting the flap valve to open during exhalation, as described above for FIGS. 4A-4D. In the example of FIGS. 5A-5C, the flap valve limiter is made from a slow-recovery elastic deformation material, or a slow-recovery shape memory material, including some foams (e.g., memory foams), that can be deformed from an original shape and slowly relax back into the original shape. This is illustrated in FIG. 5B, in which the flap valve limiters have been bent (e.g., by applying force) so that they do not prevent the flap valves from at least partially opening distally during exhalation, thereby decreasing the resistance. The delay period of such airflow resistor bypasses (e.g., the time before the nasal device restores the baseline resistance to exhalation, Re) in this example may depend on the material properties of the flap valve limiter/bypass displacers.

Any other slow-recovery material (including shape memory materials and foams) may also be used. The delay period may be adjusted by modifying the material properties (e.g., to modify elastic recovery time), as known in the art. For example, recovery time may be modified by altering the elasticity, air inclusions, etc.).

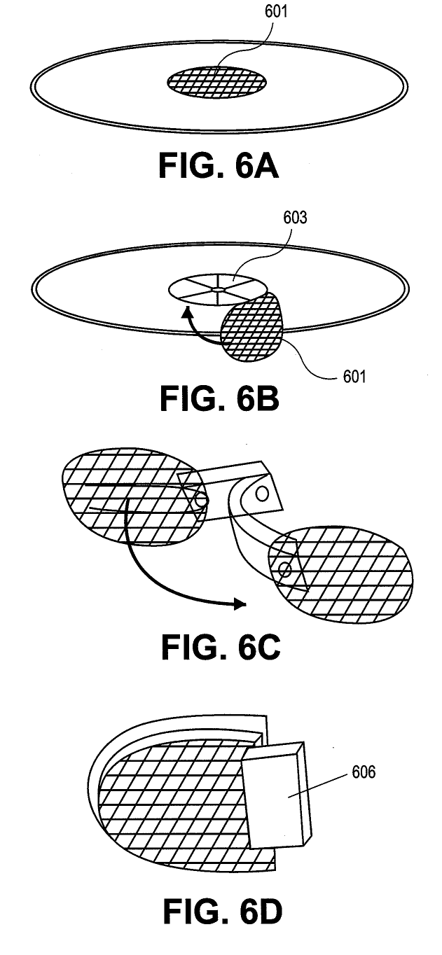

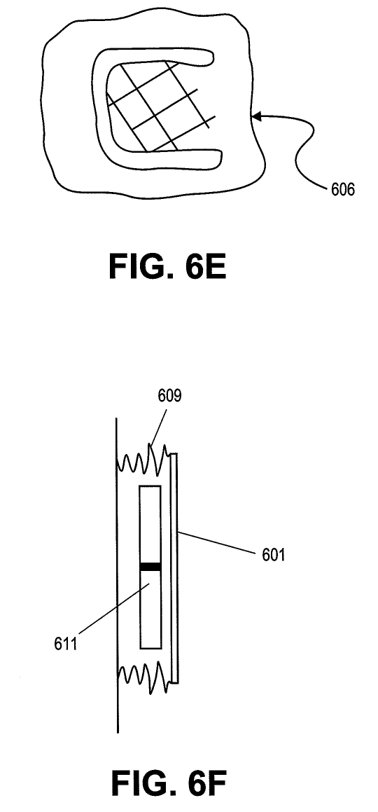

FIGS. 6A-6E illustrate different variations of airflow resistor bypasses having bypass displacers that are configured to decrease the resistance to exhalation by moving the flap valve limiter away from the flaps of the nasal device, permitting the flap valves to open somewhat (or completely) during exhalation. FIG. 6A shows a distal view of a nasal device (e.g., the side of the device facing outwards if the device is worn by a subject). As described above, this nasal device includes a flap valve limiter 601 that normally prevents the flaps of the flap valve 603 (visible in FIG. 6B) from opening during exhalation. In this example, the airflow resistor bypass is a bypass displacer configured to move the flap valve limiter 601 away from the flap, and includes a hinged region. The hinge is a bypass displacer, and may be made of a material having a slow-recovery elastic deformation, or a slow-recovery shape memory material, as mentioned above. FIG. 6C illustrates another variation of a bypass displacer including a hinge that connects to the flap valve limiter 601. Bending the hinge causes the flap valve limiter to permit the flap valve(s) to open during exhalation, decreasing the resistance to exhalation. FIGS. 6D and 6E show other variations of bypass displacers including a hinge region 606.

In FIG. 6F, an airflow resistor of a nasal device including an airflow resistor bypass is shown in cross-section. In this variation, the flap valve limiter attached to a bypass displacer that is movable, away from the flap valves 611, by extending the collapsible sleeve 609. The bypass displacer in this example includes the collapsible sleeve 609. The collapsible sleeve 609 can then slowly collapse back as shown in FIG. 6F so that the flap valve limiter 601 is once again adjacent the flap valves 611. The delay period is the time it takes the collapsible sleeve to restore the position of the flap valve limiter adjacent to the flap valve 611. The collapsible sleeve can be configured to an appropriate delay period. For example a spring or other return bias may be used to slowly return the collapsible sleeve (e.g., in seconds, minutes, etc.) back to the collapsed state during which the nasal device operates normally, and has a high resistance to exhalation compared to inhalation. In this variation the collapsible sleeve can be grasped when the subject is wearing the device, so that the user can activate or trigger the airflow resistor bypass by pulling to extend the collapsible sleeve, so that the collapsible tab acts as a pull tab. Alternatively, an additional tab, handle, or other grasping structure may be included as the trigger or activator for the airflow resistor bypass.

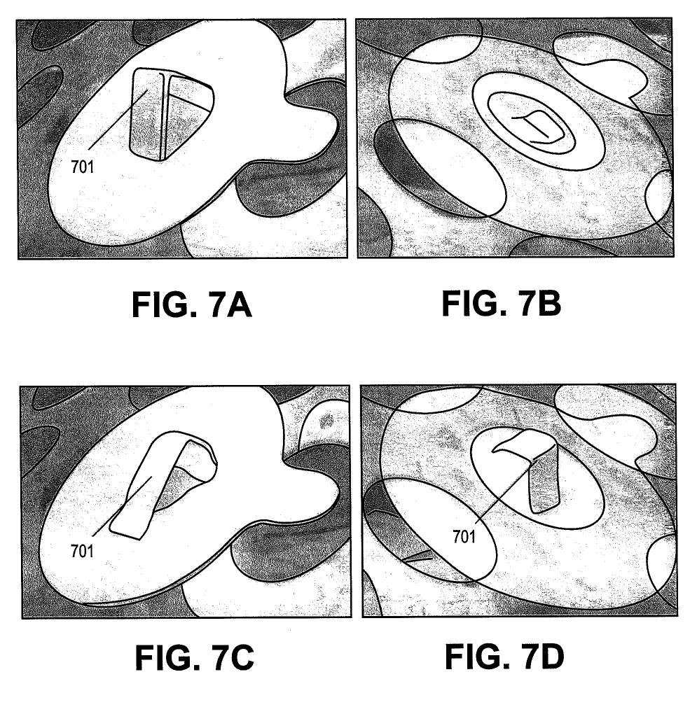

FIGS. 7A-7D illustrate another variation of an airflow resistor bypass in which a bypass displacer disrupts the closing of the flap valve by holding the flap valve open during exhalation. FIG. 7A shows the distal side of the nasal device including an airflow resistor bypass (e.g., the side facing away from a subject when the device is worn), and FIG. 7B shows the proximal side of the nasal device. The airflow resistor bypass includes the push-tab bypass displacer 701 that projects from the distal side of the nasal device. The airflow resistor bypass can be activated by pushing in the bypass displacer 701 so that it extends into the passageway of the device (the opening into which the flap valves are located). The body of the push tab displaces the flap valves proximally, preventing them from closing during exhalation, and decreasing the resistance to exhalation. This is illustrated in FIG. 7C, which show the push tab from the distal face of the nasal device, and FIG. 7D, which shows the push tab projecting through the flap valves, holding them open. The push tab slowly resumes its original position shown in FIG. 7A.

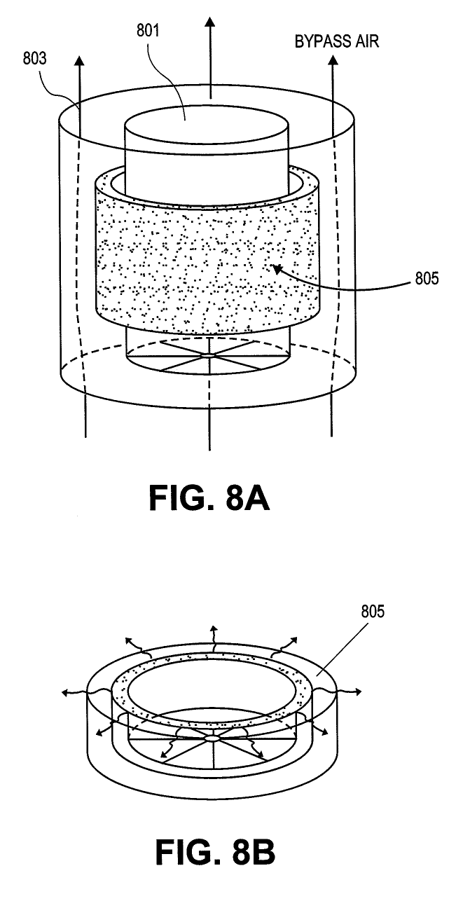

FIGS. 8A to 11C illustrate variations of nasal devices including airflow resistor bypasses having a bypass channel that creates a temporary route for airflow, thereby reducing the resistance during exhalation. For example, in FIGS. 8A and 8B, the central passageway that is regulated by an airflow resistor 801 is surrounded by a bypass channel 803, a passageway through which air can flow freely when the airflow resistor bypass is active (as it is shown in FIG. 8A). A bypass occluder 805 is positioned in the bypass channel 803. A bypass occluder is a structure that occludes a bypass channel and can be removed or reduced to open the bypass channel. The airflow resistor bypass is inactivated when the bypass occluder 805 expands to close the bypass channel 803, as shown in FIG. 8B. The bypass occluder can be compressible material, such as foams or other materials having a slow-recovery to elastic deformation. In some variations the bypass occluder is at least partially made of a shape memory material that transitions back to its original shape after the delay period. In some variations (particularly single-use variations) the bypass occluder is a material that swells when exposed to moisture. Thus, after the nasal device is applied to the subject, moisture from respiration will gradually cause the bypass occluder to expand and block off the bypass channel.

As described in further detail below, the airflow resistor bypass may form a bypass channel or channels that decrease the resistance to exhalation when the airflow resistor bypass is activated. In some variations, the airflow resistor bypass is formed of a porous material that gradually becomes blocked (e.g., occluded) by exhaled water vapor once the device is applied to a subject. Thus, the device may have an increasing resistance to exhalation after the device is applied. For example, the airflow resistor bypass may include a material such as porex that forms pores/channels through the nasal device. These pores may clog as water vapor condenses on them. In some variations the valve body is formed of a porous material such as porex. Such ‘cloggable’ variations may be single-use or multi-use.

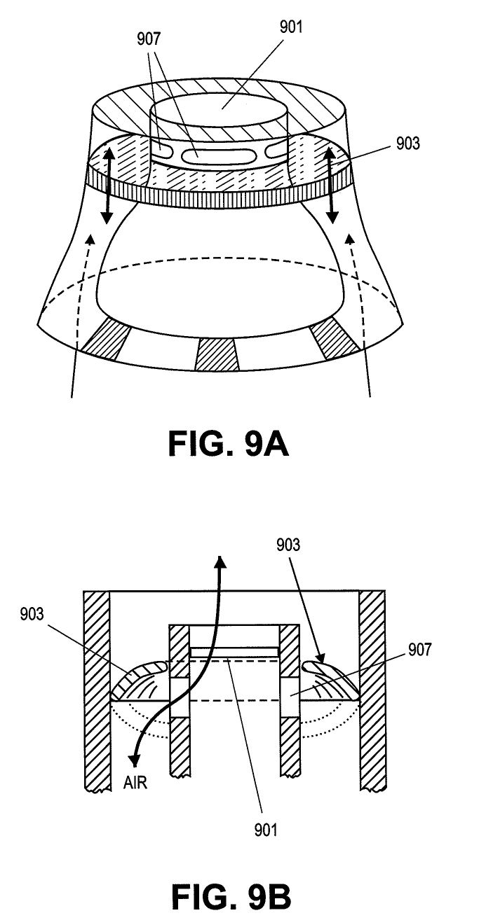

FIGS. 9A and 9B illustrate another variation in which the bypass occluder 903 is a ring that can be pushed into a first configuration that allows air to pass around the central airflow resistor 901 though the plurality of bypass channels or openings 907 formed around the periphery of the passageway in which the airflow resistor 901 is located. The bypass occluder 903 can then slowly move back up to cover the bypass channels, as shown in FIG. 9B, so that the majority of airflow again passes through the airflow resistor 901.

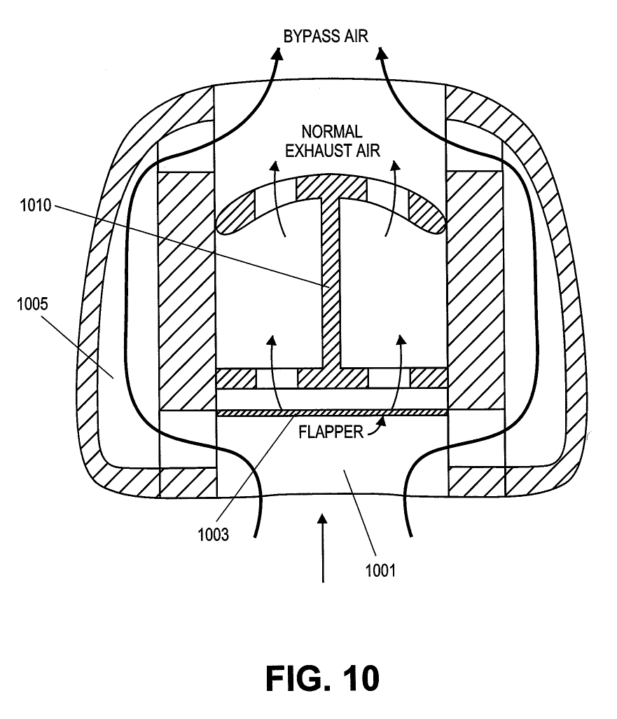

FIG. 10 is another example of a nasal device having an airflow resistor bypass configured similarly to the device shown in FIGS. 9A and 9B. In this example the bypass channel 1005 surrounds the central passageway 1001 which is occluded by the airflow resistor 1003. The airflow resistor is movable in the channel, and includes a slider 1010. The slider forms part of the airflow resistor bypass, acting as the bypass occluder. In the first position, the slider 1010 has been displaced slightly upwards (as shown in FIG. 10) and airflow may pass through the bypass channel 1005, as shown. However, the slider 1010 may be biased generally downwards (reference to FIG. 10), so that over the course of the delay period, the slider returns to the downwards position, closing off the bypass channel 1005 and inactivating the airflow resistor bypass. A spring or other mechanical bias may be used to return the slider to occlude the bypass channel, and the delay period can be determined by the force of the return bias (e.g., spring) and the frictional forces acting on the slider (or other bypass occluder).

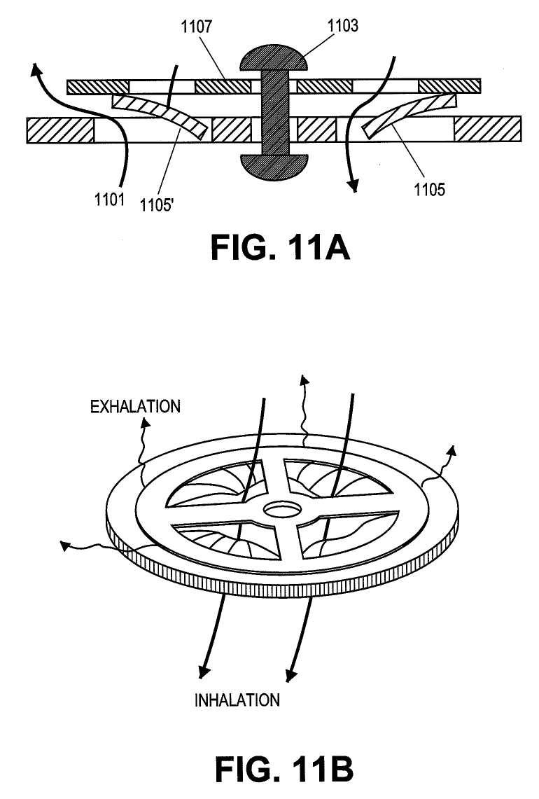

FIGS. 11A-11C show an alternative variation of an airflow resistor bypass for a nasal device. In this variation, the airflow resistor bypass includes both a bypass channel 1101 and a bypass displacer 1103. The bypass channel 1101 is normally occluded by the base of the airflow resistor, and the resistance to exhalation is determined by the airflow resistor and any leak pathways. When the airflow resistor bypass is activated (as shown in the cross-section in FIG. 11A), the airflow resistor 1107 (including flap valves 1105, 1105′) is displaced by the bypass displacer 1103, lifting the airflow resistor away and exposing the bypass channel 1101. FIG. 11B shows a perspective view of the distal side of nasal device (facing away from a subject wearing the device).

As mentioned, any of the airflow resistor bypasses described above may include a bypass timer that determines the delay period for the airflow resistor bypass configured to automatically inactivate. The delay timer may be an active timer, for example, having timing circuitry that is coupled to the airflow resistor bypass to trigger activation/deactivation of the airflow resistor bypass. Thus, a delay timer may be coupled to a driver for actuating and/or sustaining the airflow resistor bypass (e.g., a mechanical driver such as a piezo drover, magnetic driver, etc.). The delay period may also be determined by the material properties and/or structure of the airflow resistor bypass, and particularly the structure and/or materials of the bypass occluder or bypass displacer. In some variations, the delay period is determined by the rate that the material forming at least a portion of the airflow resistor bypass recovers from elastic deformation. Slow-recovery materials (e.g., materials having a slow recovery time responding to elastic deformation) are particularly useful. A slow recovery time to elastic deformation is typically on the order of seconds, minutes and hours. In some variations, the delay period is based on the transition time of a shape-memory material, such as a shape-memory alloy or polymer. For example, an airflow resistor bypass may include a bypass occluder or a bypass displacer that is made of a shape memory material having a slow transition (also referred to as recovery) time.

The delay period of the airflow resistor bypass may be preset (e.g., to approximately 15 minutes, 30 minutes, 1 hour, etc.), or it may be variable, or greater than some threshold time. In some variations, the delay period is adjustable. For example the delay period may depend on the force applied by the user to trigger or activate the airflow resistor bypass.

The delay period may also be determined by subject-dependent factors. For example, the delay period may be related to the number of exhalations and/or inhalations through the nasal device, an increase/decrease in pressure through the nasal device, the humidity that the nasal device is exposed to, or the like. For example, a portion of the airflow resistor bypass may be made from a humidity-responsive shape memory material that is capable of being deformed, storing an amount of shape deformation, and recovering at least a portion of the shape deformation when exposed to a humid environment. See, e.g., U.S. Pat. No. 6,592,995, herein incorporated by reference in its entirety. Shape-memory materials sensitive to humidity may be particularly useful in single-use airflow resistor bypass devices, in which the humidity of the subject's breath slowly returns the airflow resistor bypass to the inactive state from an initially active state when the device is first placed in communication with a nasal passage.

### Activation of an Airflow Resistor Bypass

An airflow resistor bypass for a nasal device may be automatically activated (e.g., when first worn, or when the pressure through the nasal device exceeds some threshold value), or manually activated. An airflow resistor bypass may be manually activated by a control or trigger (activator). A trigger may be controlled by the subject (e.g., a subject wearing the device). In some variations, activation of the airflow resistor bypass may be triggered by a person not wearing the device. Thus, an airflow resistor bypass may include a manual trigger such as a button, tab, pull, switch, dial, or the like. In some variations, the airflow resistor bypass is activated by an activation tool. An airflow resistor bypass may be configured to be activated by pushing a portion of the airflow resistor bypass (e.g., a button, tab, etc.), by pulling a portion of the airflow resistor bypass (e.g., tab, handle, etc.), by rotating a portion of the airflow resistor bypass (e.g., knob, dial, etc.), by pinching a portion of the airflow resistor bypass, or by any other appropriate method. The control may be located anyhere on the nasal device, and may communicate with the airflow resistor bypass.

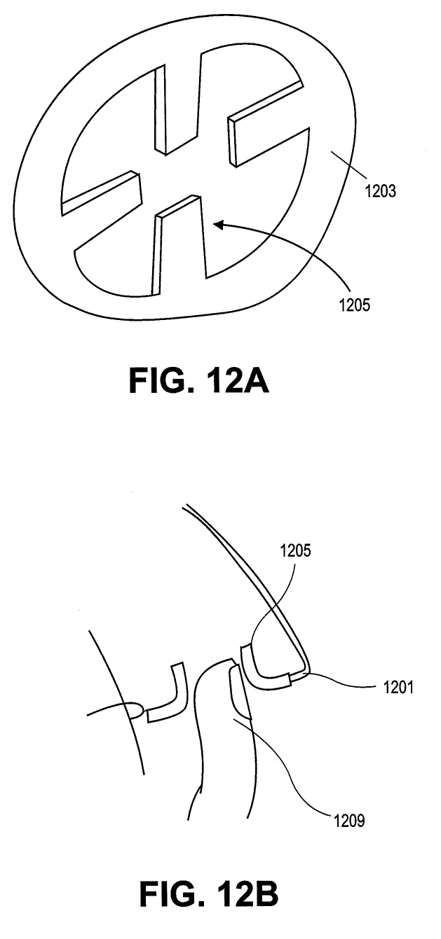

FIG. 12B illustrates manual activation of an airflow resistor bypass in a nasal device 1201 worn by a subject. In this example, the nasal device 1201 includes an airflow resistor bypass 1203 that is includes flap valve limiters 1205 that are configured as bypass displacers. The bypass displacers are formed of a material having a slow recovery time (e.g., a memory foam having a slow recovery from elastic deformation). FIG. 12A shows the distal face of this airflow resistor bypass in the inactivated state, in which the bypass displacers function as flap valve limiters that prevent the opening of the flap valve distally during exhalation. In FIG. 12B the airflow resistor bypass is triggered by pushing in on the bypass displacers so that they bend inwards, opening the flap valve. Because the bypass displacers are made of a slow-recovery material, they hold the valve in the open position during both exhalation and inhalation for a delay period. The delay period is equivalent to the time it takes the bypass displacers to recover from the elastic deformation caused by deforming them. Thus, the delay time may depend upon the material used to form the bypass displacers, the shape of the bypass displacers, and the force used to activate them by pushing them in.

The airflow resistor bypass in FIG. 12B is manually activated by inserting a finger 1209 into the nasal device to open the airflow resistor bypass. In this example, the finger 1209 activates the airflow resistor bypass, however an activation tool or element may be used instead. An activation tool may activate an airflow resistor bypass. Activation tools may be used to push, pull, heat, energize, magnetize, or otherwise activate an airflow resistor bypass, as appropriate. In some variations an activation tool is a probe that extends into the nasal device to engage a portion of the airflow resistor bypass.

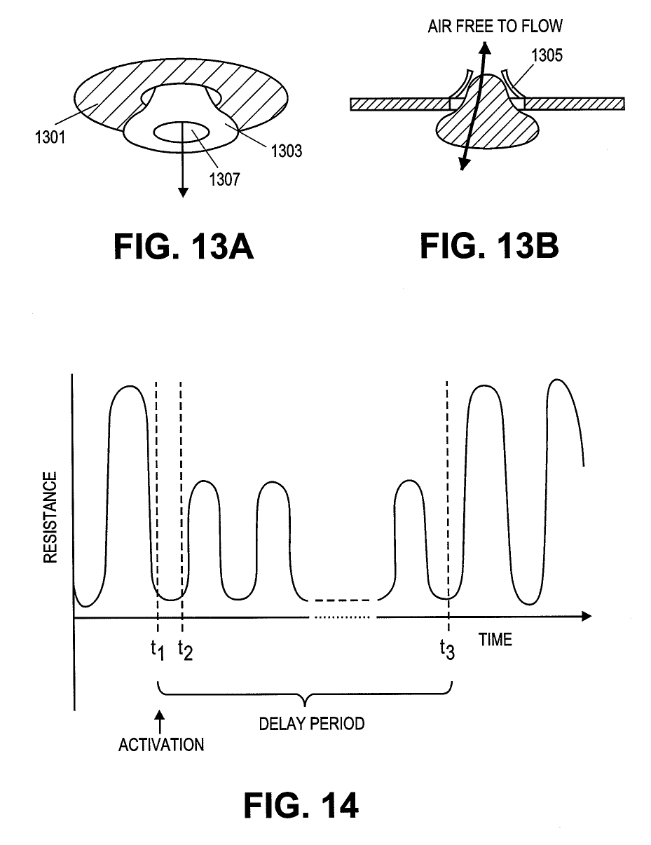

In FIG. 13A, an airflow resistor bypass 1303 has been inserted into the nasal device 1301 activating the airflow resistor bypass by displacing the flap valves 1305. This is visible in the side view in FIG. 13B. In this example, the airflow resistor bypass is active while inserted into the nasal device, since the airflow resistor bypass 1303 includes a bypass passageway 1307 through which respiration may occur while the airflow resistor bypass is inserted in the nasal device. After the airflow resistor bypass has been removed, the nasal device may resume normal operation. In some variations the nasal device also includes an airflow resistor bypass that is not removable, but it part of the nasal device.

As mentioned above, in some variations the airflow resistor bypass is activated by the force of exhalation through the device. In other variations the airflow resistor bypass may be responsive to the position of the nasal device. For example, the airflow resistor bypass may be activated by the orientation (or a change in orientation) of the nasal device. An airflow resistor bypass may include a ballast element that can trigger the airflow resistor bypass when the subject is upright, or when the subject moves upright from a prone position.

### EXAMPLES

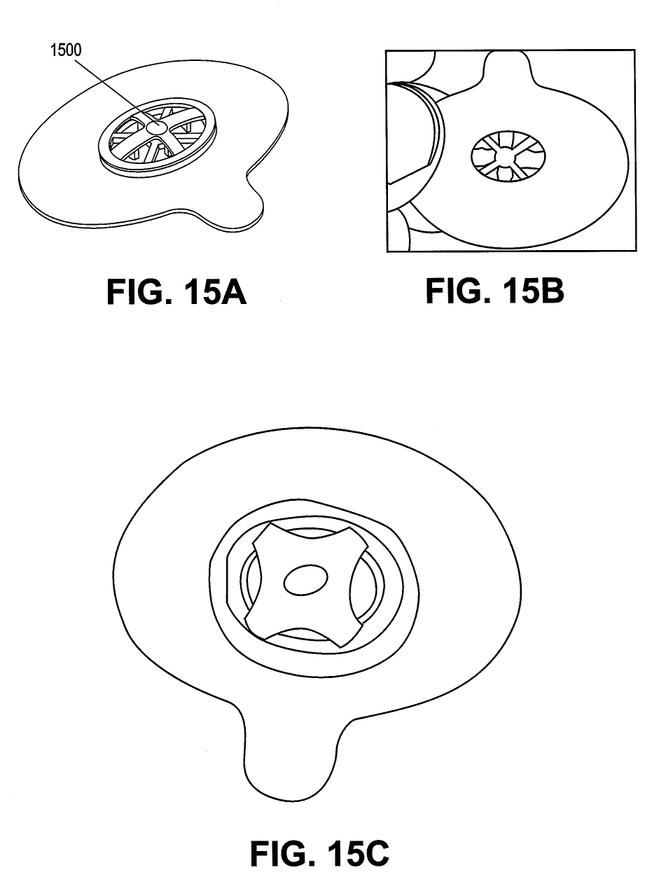

FIGS. 15A-27B illustrate additional variations of airflow resistor bypasses and nasal devices including airflow resistor bypasses. These examples represent only some of the ways of controllably (e.g., activatably) decreasing the resistance to exhalation for some delay period td, before resuming baseline activity. Other solutions (including complex mechanical, electrical, electromagnetic, and electromechanical solutions) are possible, and intended to be encompassed by this invention.

FIGS. 15A to 20B illustrate airflow resistor bypasses having one or more bypass displacers for disrupting a flap-valve airflow resistor that can be activated by a button on the bottom of the nasal device (the side facing away from the nasal passage when the device is worn by a subject). FIGS. 15A-18 and 20A-20B are airflow resistor bypasses with adhesive buttons. In these examples the adhesive properties of the adhevise acts as the delay timer.

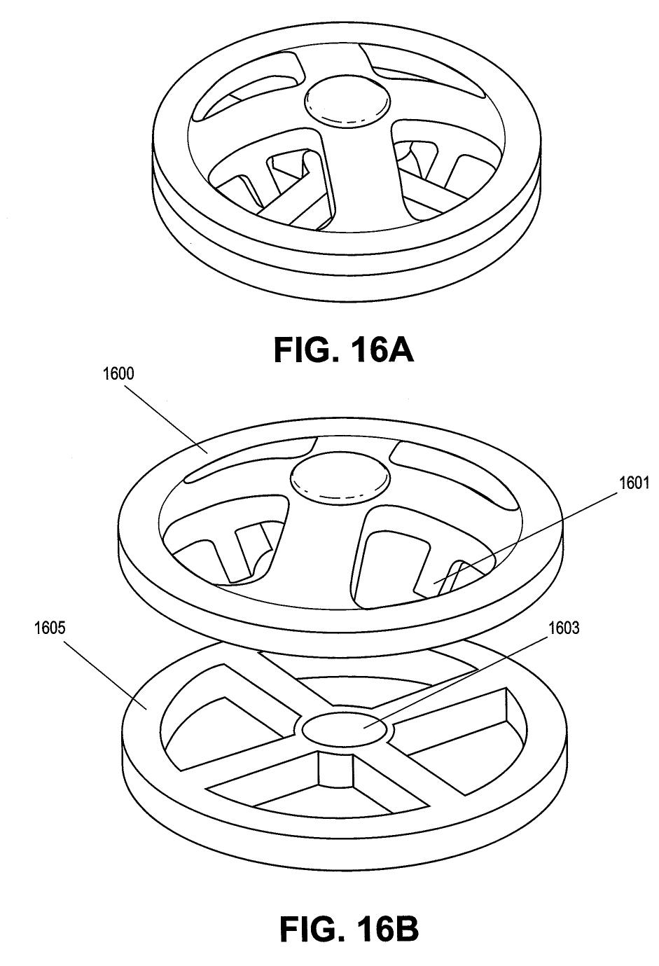

For example, FIG. 15A shows a perspective view of a nasal device including an airflow resistor bypass 1500. This airflow resistor bypass includes a dome-shaped button over one side of the passageway of the nasal device. The dome is formed of (in this example) two cross-connecting arced struts that meet at the center of the passageway. The underside (facing the passageway and the airflow resistor) of each strut includes two bypass displacers (posts) that can be pushed (by pushing on the button) into the airflow resistor to activate the airflow resistor bypass. When the airflow resistor bypass is activated, the bypass displacers push against the flap valves and prop them open. The button will remain pushed (and the bypass displacers will continue to disrupt the airflow resistor) based on the adhesion between a patch of adhesive on the underside of the struts and the portion of the nasal device near the airflow resistor, as illustrate din FIG. 16B. FIG. 15B shows a top view of the nasal device (on the opposite side from the button).

The button may be made of an elastomeric or shape-recovery material (including plastics, metals, etc.), so that after being deformed, it can return to the dome shape once the adhesive has released. Since the elastic properties of the material forming the dome may act against the adhesive holding the dome collapsed, the material used, as well as the shape of the dome, may partially determine the duration of the delay period.

FIG. 15C shows another variation of an airflow resistor bypass similar to that shown in FIGS. 15A and 15B. In this example, the dome includes a tactile marker (a bump) in the center that can help guide the user where to push against the dome. The dome may be configured to provide additional tactile feedback. For example, the dome may be a “snap dome” that clicks or otherwise provides palpable feedback when pushed down.

FIG. 16 is shows another variation of an airflow resistor bypass including a bump on the button that helps guide the user on where to press to activate the airflow resistor bypass. FIG. 16B shows an exploded view of the airflow resistor bypass button in FIG. 16A. In this example, the bypass displacers (legs 1601) are visible on the airflow resistor bypass 1600, and an adhesive pad 1603 is also visible on the outer surface of the flap valve limiter of the airflow resistor 1605. This adhesive pad may mate with an adhesive mating surface (which may also include an adhesive) on the dome of the airflow resistor bypass.

FIGS. 17A and 17B illustrate another variation of an adhesive airflow resistor bypass, in which the dome or button portion of the airflow resistor bypass includes only a single arced strut to which the bypass displacer(s) are attached.

The exploded view of a nasal device with an airflow resistor bypass shown in FIG. 18 illustrates one variation for assembling such a nasal device. In this example, a liner 1801 covers the proximal (subject-facing) side of the adhesive substrate 1807, to which an alignment cone 1805 is secured between the adhesive substrate and a ring of double-sided adhesive 1803. The adhesive holdfast is flap valve 1809 and flap valve limiter 1813 that are secured between the adhesive holdfast and a double sided adhesive 1811. The airflow resistor bypass (snooze button 1815) is secured over the flap valve limiter 1813 between a single-sided adhesive 1817 and the double-sided adhesive 1811 and/or the adhesive substrate 1807.

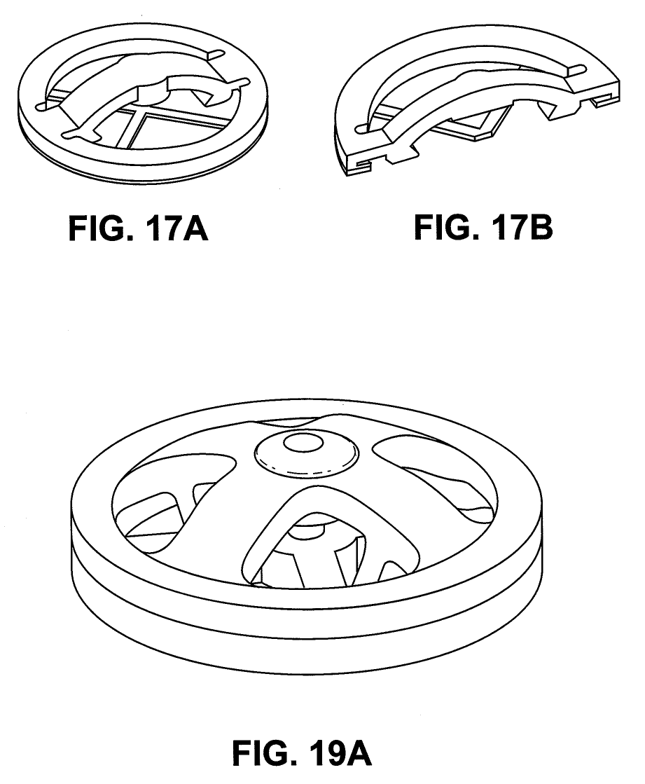

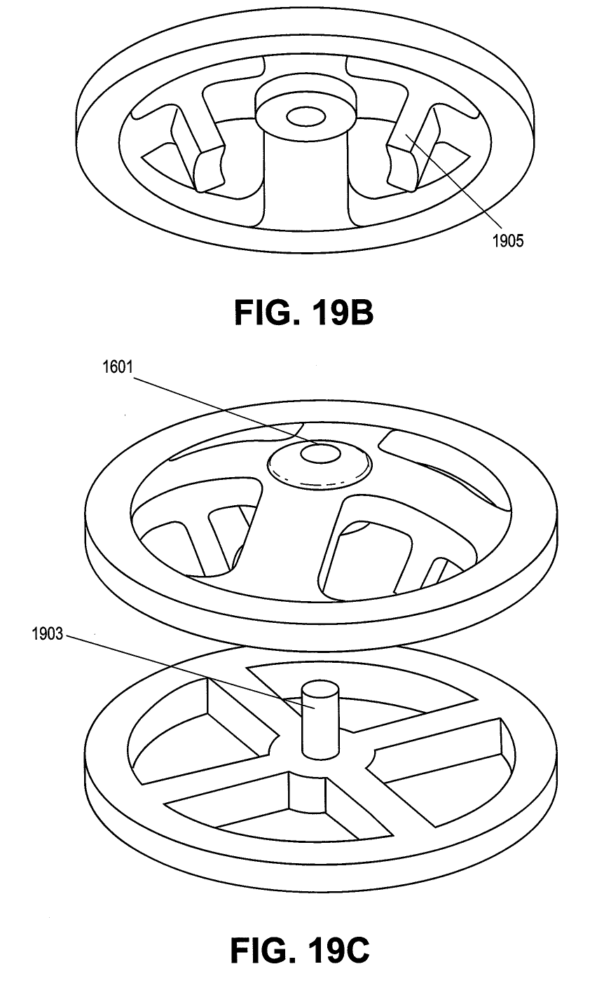

FIGS. 19A-19C illustrate another variation of an airflow resistor bypass including a dome that may act as an activation button. In this variation, the airflow resistor bypass uses an adhesive material within a central channel 1901 in the center of the dome. This center channel mates with a post 1903 that projects from the flap valve limiter. Shear adhesion between the post 1903 and the channel in the dome 1901 may determine the delay period. Thus, these elements may act as part of the mechanical delay timer, determining how long the airflow resistor bypass remains active. The airflow resistor bypass also includes two or more bypass displacers 1905 projecting from the arc(s) forming the domed button, as shown in FIGS. 19B and 19C.

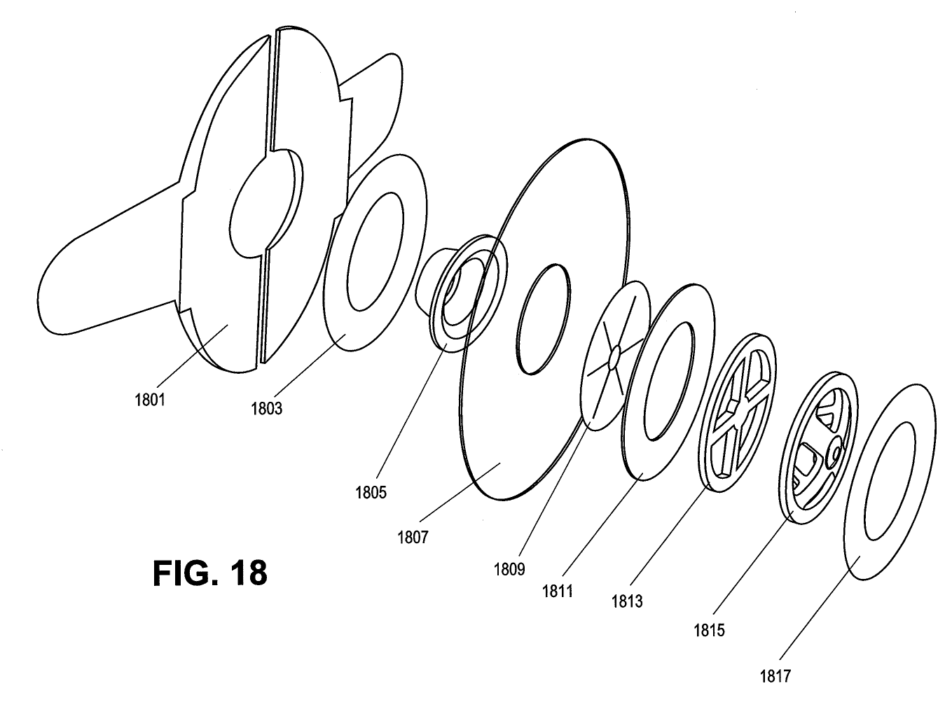

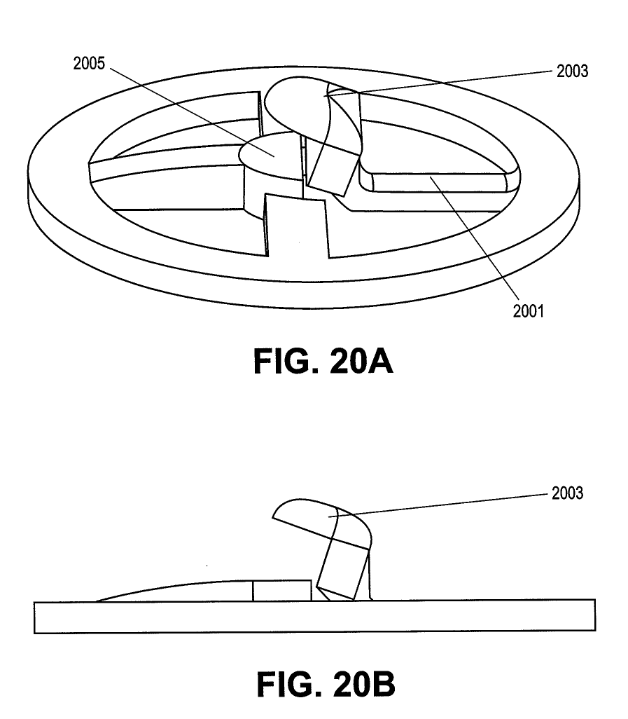

Another variation of an airflow resistor bypass including a bypass displacer in which the delay timer includes an adhesive material is shown in FIGS. 20A-20B. In this example, the flap valve limiter of an airflow resistor may be configured as an airflow resistor bypass. One arm of the flap valve limiter 2001 includes a projection 2003 that extends from the plane of the flap valve limiter. This arm is also configured as a bypass displacer. The distal end of the projection 2003 ends in a button or knob shape on the bottom surface (the surface facing away from the airflow resistor). The underside of this button includes a first adhesive mating surface (not visible) that forms part of the delay timer, and can mate with a second adhesive mating surface 2005 immediately below it (e.g., on the flap valve limiter). Pushing the button activates the airflow resistor bypass, bending the arm until the first adhesive mating surface contacts (and is releasably secured to) the second adhesive mating surface, and pushing one arm of the flap valve limiter 2001 (the arm to which the projection is attached) into the flap valve, preventing it from closing completely during exhalation. After the delay period, which may depend on the strength of the adhesive connection between the two adhesive mating surfaces, as well as the elastomeric properties of the bypass displacer, the bypass displacer returns to the inactive position, and the entire structure functions as the flap valve limiter until the next time the button is pushed. FIG. 20B is a side view of this airflow resistor bypass in the inactive state.

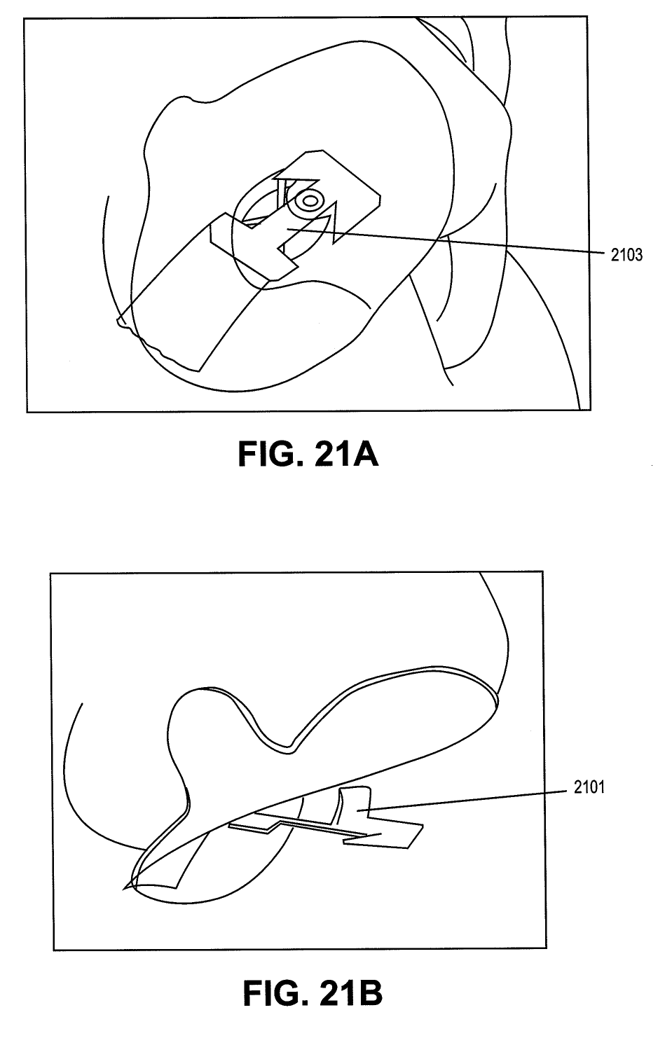

FIGS. 21A and 21B illustrate a nasal device include an airflow resistor bypass configured as a lever having an adhesive-based delay timer. In this variation, a bypass displacer (post 2101) projects from a hinged lever arm 2103. The distal end of this lever arm includes an adhesive mating surface that contacts a second adhesive mating surface (elsewhere on the nasal device). The lever arm may be biased in the open state, in which the bypass displacer is inactive, and does not interfere with the airflow resistor. This bias may be a result of a material or structural property of the lever arm (2103), or it may be biased by a spring or other structure. In some variations the hinged lever arm is a living hinge, and the material includes elastic properties that urge it back into the inactivated shape. In some variations the airflow resistor bypass also includes a metal spring or other spring element biasing the arm.

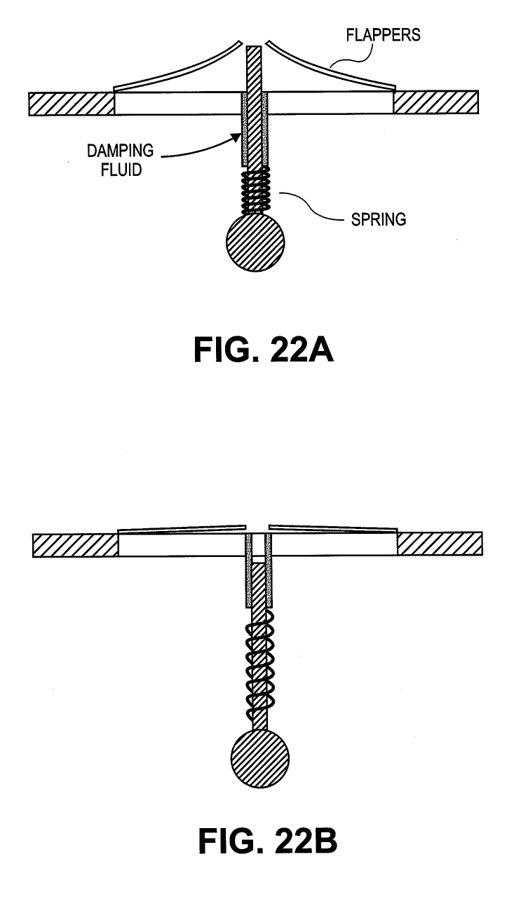

FIGS. 22A and 22B illustrate another variation of a nasal device having an airflow resistor bypass including a bypass displacer. In this example (shown here in cross-section), the delay timer includes a damping fluid that is opposed by a spring bias. The damping fluid is a visco-elastic, non-Newtonian liquid. The airflow resistor bypass is activated by pushing in the button, as shown in FIG. 22A, causing the bypass displacer to interfere with the flap valves, and compressing the spring bias. The delay period of the airflow resistor bypass is the time that it takes the linear bypass displacer to return to the inactivated position, shown in FIG. 22B.

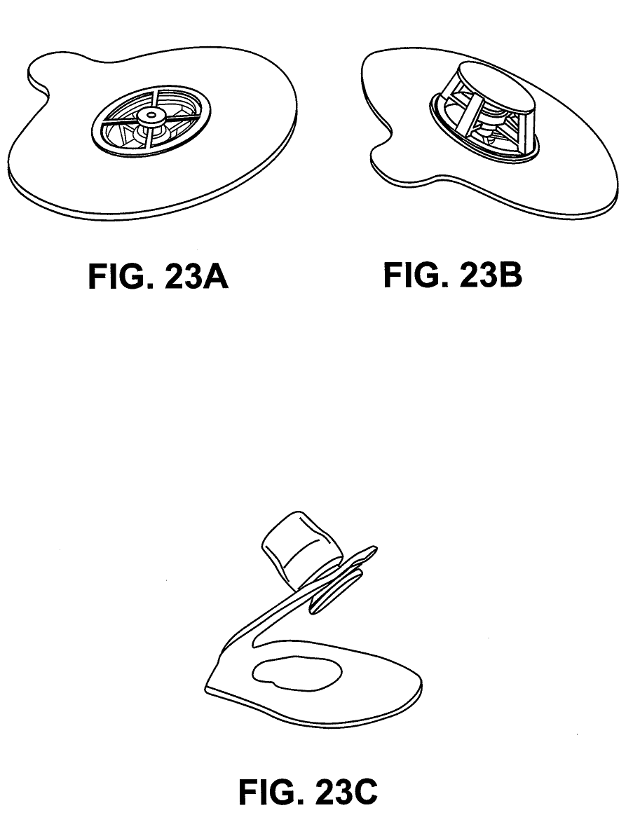

FIGS. 23A and 23B illustrate another variation of a nasal device including an airflow resistor bypass. In this example, the airflow resistor bypass includes a suction cup as part of the delay timer. Pushing in on the airflow resistor bypass button causes one or more bypass displacers to interfere with the airflow resistor, and engages a suction cup. The delay period will last as long as the suction cup is able to maintain suction.

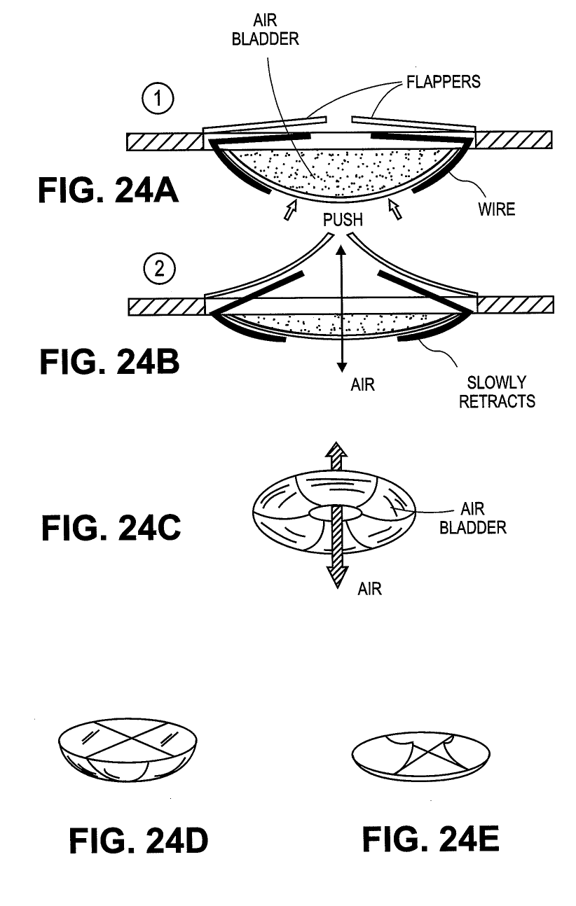

In one variation, the airflow resistor bypass includes a bladder, as shown in FIGS. 24A-24E. In this example, the air bladder is compressed to activate the airflow resistor bypass, as shown in FIG. 24B. The refilling of the air bladder returns the bypass displacers to the initial (non-interfering) position, shown in FIG. 24A. An example of an airbladder is shown in FIG. 24C. FIGS. 24D and 24E show the inner (nostril-facing) view of the airflow resistor when the airflow resistor bypass is inactivated (FIG. 24D), and when it is activated (FIG. 24E).

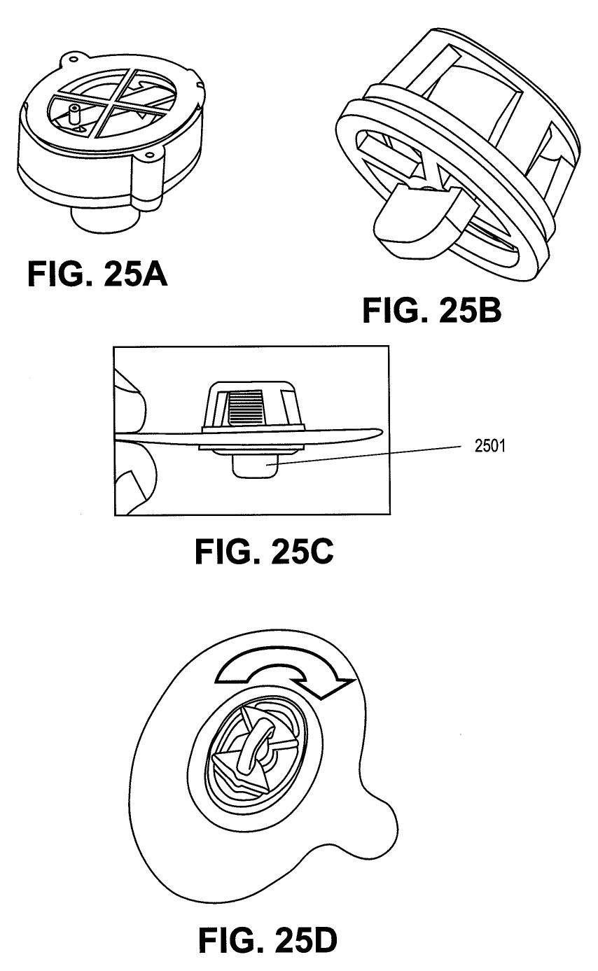

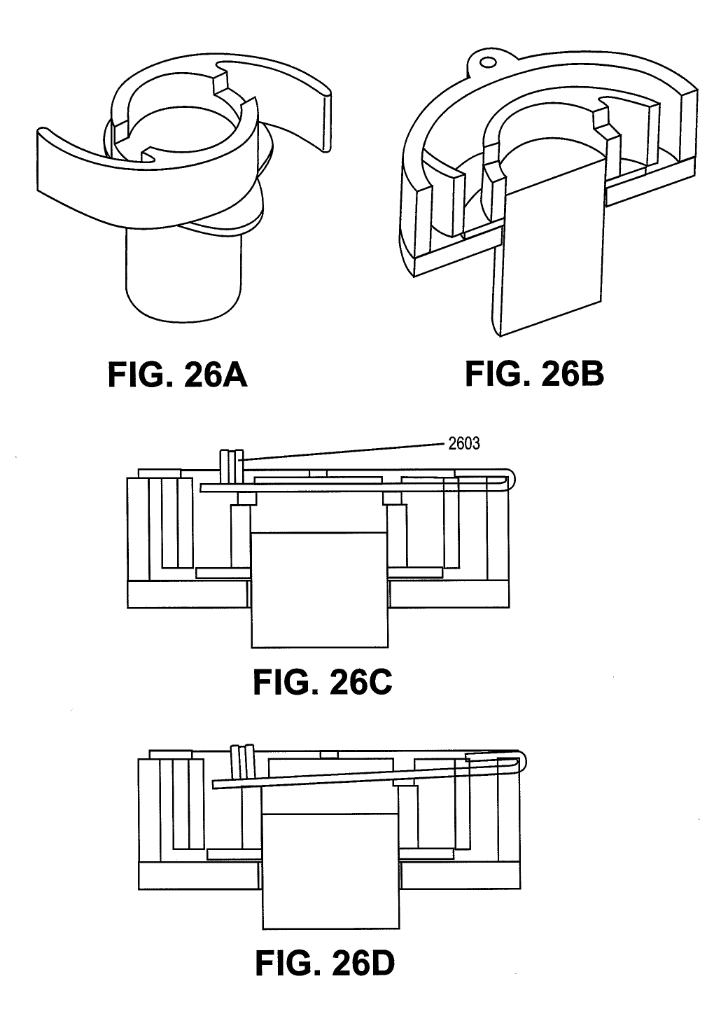

FIGS. 25A-25D show a bypass delay that is activated by rotation of a knob. In this example, the clockwise twisting of knob 2501 (shown in FIG. 25D) of the airflow resistor bypass activates the delay. FIG., 25C shows a side perspective view of this device. The operation of this device is illustrated in detail in FIGS., 26A-26D. FIG. 26A shows the dampening paddles that extend into a dampening medium, as illustrated in FIG. 26B. This dampening medium may be a visco-elastic (and non-Newtonian) fluid, as described above for FIGS. 24A and 24B. Twisting of the assembly including the paddles may elevate a bypass displacer (tab 2603 in FIG. 26C), and thereby disrupt the airflow resistor. A biasing force (e.g., spring or other bias) may oppose this, and act against the visco-elastic fluid, eventually returning the bypass displacer to the inactive position for the airflow resistor bypass, as shown in FIG. 26D in cross-section.

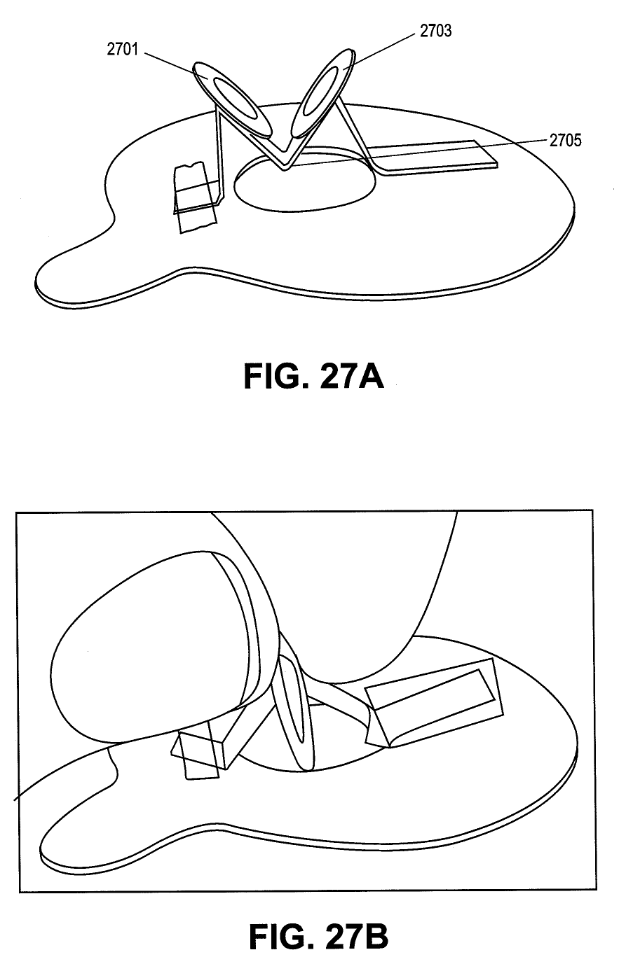

Finally, FIGS. 27A and 27B illustrate another variation of a nasal device including an airflow resistor bypass. This airflow resistor bypass may be configured as a suction-based bypass timer, or an adhesive-based bypass timer, and a bypass displacer. FIG. 27A shows the airflow resistor bypass in the inactive position. The first 2701 and second 2703 pads can be pressed together to extend the bypass displacer (hinge region 2705) into the airflow resistor, as shown in FIG. 27B. After the delay period the first and second regions release, and the hinge region forming the bypass displacer is withdrawn from the airflow resistor.

In operation, an airflow resistor bypass may be used to reduce the resistance to exhalation in any appropriate nasal device, particularly nasal respiratory devices having an airflow resistor that is configured to increase the resistance to exhalation more than inhalation. The airflow resistor bypass can be activated either manually or automatically, resulting in a reduction in the resistance to air exhaled through the device for some delay period that extends beyond the activation of the airflow resistor bypass. The airflow resistor bypass accomplishes the reduction in resistance to exhalation by opening a bypass channel and/or by modifying or disabling the airflow resistor so that additional airflow may occur during exhalation when the airflow resistor bypass is activated.

An airflow resistor bypass may include a sustained delay period that extends well past the activation or triggering event. Thus, an airflow resistor bypass is not simply a pressure release valve, because it decreases the pressure for a period of time after the triggering event. For example, if the airflow resistor bypass is triggered or activated by an increase in pressure during exhalation, the airflow resistor bypass sustains the reduction in expiratory pressure during the delay period that extends even after the pressure falls outside of the triggering range. Thus, the delay period of the airflow resistor bypass is typically uncoupled from the duration of the triggering or activation event. This is apparent in FIG. 14, which illustrates a hypothetical resistance profile for a nasal device having an airflow resistor bypass. The airflow resistor bypass is triggered at time t₁, and the triggering event (pulling a tab, squeezing the trigger, increasing pressure in the nasal device, etc.) lasts until time t₂. Thus, the duration of the triggering event, t_{trigger}, is t₂−t₁. The airflow resistor bypass activates and remains activated (decreasing the resistance to exhalation through the device) until t₃. The delay period for this airflow resistor bypass is thus t_{delay}, which is t₃−t₁ (or in some variations, t₃−t₂). In general, the delay period, t_{delay}, is much longer than the triggering event duration (t_{delay}>>t_{trigger}), and the delay period extends for some time after the triggering or activation event.

Nasal respiratory devices including airflow resistor bypasses may be used by any appropriate subject, and may be particularly useful when the subject is going to sleep. An airflow resistor bypass may be activated prior to sleeping, so that the onset of sleep occurs when the airflow resistor bypass is active.

In some variations the airflow resistor bypass is activated to allow the subject to acclimate gradually to the baseline resistance to exhalation. A nasal device may be configured so that the airflow resistor bypass is active before the subject wears the nasal device. Alternatively, the airflow resistor bypass may be activated by application of the nasal device. For example, a nasal device such as that shown in FIGS. 13A and 13B may include an activation tool already inserted into the nasal device before the device is worn. Once the nasal device is applied, the activation tool can be removed and the baseline higher resistance to exhalation will be restored. Pre-activated or activation-upon-application airflow resistor bypasses may be single-use airflow resistor bypasses. In some variations the airflow resistor bypass includes a frangible bypass displacer that is disrupted when the nasal device is first placed in communication with the subject's nasal passage. For example, the bypass displacer may be made of a wax or other removable material that initially prevents the airflow resistor from closing completely (thereby reducing resistance to exhalation) but over time melts, dissolves, or breaks, allowing full baseline activity of the airflow resistor.

Many other materials and structures may be used to achieve the airflow resistor bypass of the airflow resistor as described. This description is not intended to be limited to the structures and materials described herein, but is also intended to encompass many other materials and structures having similar properties.

Although the nasal devices described herein are configured so that (in normal operation) the resistance through the device is greater during exhalation than during inhalation, other configurations may also be used with the airflow resistor bypasses described herein. For example, a nasal device may be configured with an airflow resistor that inhibits inhalation more than exhalation, which may be used with an airflow resistor bypass configured to temporarily decrease the resistance to inhalation. While the methods and devices have been described in some detail here by way of illustration and example, such illustration and example is for purposes of clarity of understanding only. It will be readily apparent to those of ordinary skill in the art in light of the teachings herein that certain changes and modifications may be made thereto without departing from the spirit and scope of the invention.

What is claimed is:

1. A nasal respiratory device generally configured to provide an increased resistance to nasal exhalation more than inhalation, the device comprising:
an opening configured to communicate with a nasal passage;
an airflow resistor in communication with the opening, wherein the airflow resistor is configured to increase the resistance to air exhaled through the opening more than air inhaled through the opening; and
an airflow resistor bypass configured to suspend or reduce the increased resistance to exhalation applied by the airflow resistor, wherein the airflow resistor bypass is configured to be inactivated.

2. The device of claim 1, further comprising a manual trigger for activating the airflow resistor bypass, wherein operating the trigger activates the airflow resistor bypass and decreases the resistance to exhalation through the nasal device.

3. The device of claim 1, wherein the airflow resistor bypass is configured to be manually inactivated by manipulating a control.

4. The device of claim 1, wherein the airflow resistor bypass is configured to be automatically inactivated after a delay period.

5. The device of claim 4, wherein the delay period is greater than 15 minutes.

6. The device of claim 4, wherein the delay period is less than 2 hours.

7. A nasal respiratory device configured to temporarily decrease resistance to exhalation through the device, the device comprising:
an opening configured to communicate with a nasal passage;
an airflow resistor in communication with the opening, wherein the airflow resistor is configured to increase the resistance to air exhaled through the opening more than air inhaled through the opening; and
an airflow resistor bypass configured to transiently decrease the resistance to air exhaled through the opening for a delay period, after activation of the airflow resistor bypass.

8. The device of claim 7, further comprising a holdfast configured to secure the respiratory device at least partly over or within a nasal passage.

9. The device of claim 8, wherein the holdfast comprises an adhesive holdfast.

10. The device of claim 7, wherein the airflow resistor comprises a flap valve.

11. The device of claim 7, wherein the airflow resistor bypass is configured so that the delay period is greater than 5 minutes.

12. The device of claim 7, wherein the airflow resistor bypass is configured so that the delay period is greater than 15 minutes.

13. The device of claim 7, wherein the airflow resistor bypass is configured so that the delay period is less than 2 hours.

14. The device of claim 7, wherein the airflow resistor bypass comprises a bypass channel through which air may pass during exhalation during the delay period.

15. The device of claim 14, further comprising a bypass occluder configured to occlude the bypass channel when the airflow resistor bypass is inactive.

16. The device of claim 7, wherein the airflow resistor bypass comprises a delay timer that determines the delay period.

17. The device of claim 7, wherein the airflow resistor bypass comprises a bypass displacer configured to displace or disable at least a portion of the airflow resistor during the delay period.

18. The device of claim 17, wherein the airflow resistor bypass comprises a post or projection configured to hold the airflow resistor at least partially open activation of the airflow resistor bypass.

19. The device of claim 7, wherein the airflow resistor bypass further comprises a trigger for activating the airflow resistor bypass

20. The device of claim 19, wherein the trigger comprises a push or pull tab.

21. A nasal respiratory device configured to decrease resistance to exhalation through the device for a delay period, the device comprising:
an opening configured to communicate with a nasal passage;
an airflow resistor in communication with the opening, wherein the airflow resistor is configured to increase the resistance to air exhaled through the opening more than air inhaled through the opening; and
an airflow resistor bypass including a bypass channel and a bypass occluder, wherein the airflow resistor bypass is configured to transiently decrease the resistance to air exhaled through the opening for a delay period after activation of the airflow resistor bypass by opening the bypass channel through which air may be exhaled.

22. The device of claim 21, further comprising a holdfast configured to secure the respiratory device at least partly over or within a nasal passage.

23. The device of claim 22, wherein the holdfast comprises an adhesive holdfast.

24. The device of claim 21 wherein the airflow resistor comprises a flap valve.

25. The device of claim 21, wherein the airflow resistor bypass is configured so that the delay period is greater than 5 minutes.

26. The device of claim 21, wherein the airflow resistor bypass is configured so that the delay period is less than 2 hours.

27. A nasal respiratory device configured to temporarily decrease resistance to exhalation through the device for a delay period, the device comprising:
an opening configured to communicate with a nasal passage;
an airflow resistor in communication with the opening, wherein the airflow resistor is configured to increase the resistance to air exhaled through the opening more than air inhaled through the opening; and
an airflow resistor bypass including a bypass displacer, the airflow resistor bypass configured to transiently decrease the resistance to air exhaled through the opening for a delay period after activation of the airflow resistor bypass by engaging at least a portion of the airflow resistor with the bypass displacer.

28. The device of claim 27, further comprising a holdfast configured to secure the respiratory device at least partly over or within a nasal passage.

29. The device of claim 28, wherein the holdfast comprises an adhesive holdfast.

30. The device of claim 27, wherein the airflow resistor comprises a flap valve.

31. The device of claim 27, wherein the airflow resistor bypass is configured so that the delay period is greater than 5minutes.

32. The device of claim 27, wherein the airflow resistor bypass is configured so that the delay period is less than 2 hours.

33. A method of temporarily decreasing resistance to exhalation through a nasal respiratory device, the nasal respiratory device having an airflow resistor configured to be positioned in communication with a nasal passage, wherein the airflow resistor is further configured to increase the resistance to exhalation through the device more than inhalation through the device, the method comprising:
activating an airflow resistor bypass on the airflow resistor; and
decreasing the resistance to air exhaled through the device for a delay period after activation of the airflow resistor bypass.

34. A method of temporarily decreasing the resistance to exhalation through a nasal respiratory device, the method comprising:
applying a nasal device at least partially over the subject's nose without covering the subject's mouth, wherein the nasal device includes an opening configured to communicate with the subject's nasal passageway and an airflow resistor configured to inhibit exhalation through the nasal device more than inhalation through the nasal device; and
decreasing the resistance to exhalation through the nasal device, wherein the decrease in resistance to exhalation is sustained for a delay period after triggering.

35. The method of claim 34, further comprising restoring the resistance to exhalation through the nasal device.

36. The method of claim 34, wherein the decrease in resistance through the nasal device is triggered by activating an airflow resistor bypass on the airflow resistor.

37. The method of claim 34, wherein the resistance is decreased by temporarily opening a bypass channel through which air may be exhaled.

38. The method of claim 34, wherein the resistance is decreased by engaging at least a portion of the airflow resistor with a bypass displacer, thereby temporarily disabling the airflow resistor.

39. A method of acclimating a subject to a nasal respiratory device, the method comprising:
applying a nasal device at least partially over the subject's nose without covering the subject's mouth, wherein the nasal device includes an opening configured to communicate with the subject's nasal passageway and an airflow resistor configured to inhibit exhalation through the nasal device more than inhalation through the nasal device; and
gradually increasing the resistance to exhalation through the nasal device.

40. A nasal respiratory device having an activatable resistance to exhalation through the device, the device comprising:
an opening configured to communicate with a nasal passage;
an airflow resistor in communication with the opening, wherein the airflow resistor is configured to increase the resistance to air exhaled through the opening more than air inhaled through the opening; and
a control configured to activate and to inactivate the application of the increased resistance to air exhaled through the opening more than air inhaled through the opening by the airflow resistor.

41. A method of controlling the resistance to exhalation through a nasal respiratory device, the nasal respiratory device having an airflow resistor configured to be positioned in communication with a nasal passage, wherein the airflow resistor is configured to increase the resistance to exhalation through the device more than inhalation through the device, the method comprising:
operating a control on the nasal device to permit the airflow resistor to inhibit exhalation through the device more than inhalation through the device; and
operating the control on the nasal device to prevent the airflow resistance from inhibiting exhalation through the device more than inhalation through the device.
